(19) United States
(12) Patent Application Publication
(10) Pub. No.: US 2025/0129114 A1
(43) Pub. Date: Apr. 24, 2025

(54) Solid Phase Peptide Synthesis (SPPS) Processes and Associated Systems

(71) Applicant: CEM Corporation, Matthews, NC (US)

(72) Inventors: Jonathan McKinnon Collins, Charlotte, NC (US); Sandeep K. Singh, Charlotte, NC (US); David Lee Herman, Charlotte, NC (US); Levi Joshua Tubb, Charlotte, NC (US)

(21) Appl. No.: 18/689,968

(22) PCT Filed: Sep. 16, 2022
(86) PCT No.: PCT/US2022/043806

§ 371 Date: Mar. 7, 2024

Related U.S. Application Data
(60) Provisional application No. 63/245,674, filed on Sep. 17, 2021.
(60) Provisional application No. 63/401,349, filed on Aug. 26, 2022.

(51) Int. Cl. C07K 1/04; C07K 1/107

(52) U.S. Cl. CPC C07K 1/045; C07K 1/107

(57) ABSTRACT

A process for deprotecting an amino acid during solid phase peptide synthesis includes removing a protecting group of a protected amino acid with a deprotecting agent in a reaction vessel, during which at least a portion of the deprotecting agent volatizes or evaporates into an upper interior portion of the reaction vessel. The process further includes directing an inert gas through the interior of the reaction vessel to purge volatized deprotecting agent from the interior of the reaction vessel. The present disclosure also relates to a solid phase peptide synthesis system for deprotecting an amino acid.

### CROSS-REFERENCE TO PRIORITY APPLICATIONS

This application claims priority to, and incorporates by reference the entire disclosure of, U.S. Provisional Patent Application No. 63/245,674, filed Sep. 17, 2021.

This application also claims priority to, and incorporates by reference the entire disclosure of, U.S. Provisional Patent Application No. 63/401,349, filed Aug. 26, 2022.

### FIELD

The present disclosure relates to solid phase peptide synthesis processes and associated systems.

### BACKGROUND

Since its inception in 1963, Solid Phase Peptide Synthesis has been a major enabling tool for peptide synthesis. J. Am. Chem. Soc. 1963, 85, 14, 2149-2154. Solid phase peptide synthesis (also “SPPS”) is a process used to chemically synthesize peptides on solid supports, such as a solid phase resin. SPPS includes a series of steps that are repeated to couple amino acids to form a peptide.

In a SPPS process, an initial amino acid is attached to a solid support by a linking group usually on the carboxyl or C-terminus. The initial amino acid also includes a protecting group, usually on the amine or N-terminus, to protect from unwanted reactions.

A deprotecting agent removes the protecting group (the initial amino acid is “deprotected”) so that a second amino acid via its acid group can be coupled to the amine group on the initial acid. The second (and succeeding) acids are also initially protected.

Thus, the general sequence of solid phase peptide synthesis is to deprotect, couple, and repeat until the desired peptide is completed, following which the completed peptide is cleaved from the solid phase resin.

SPPS dramatically simplified solution-based peptide synthesis and provided a framework for building a peptide chain one amino acid at a time through repetitive deprotection and coupling steps. This was due to its straightforward isolation by simple filtration as opposed to more tedious extraction processes after each deprotection and coupling step.

Conventionally, SPPS processes require multiple washing steps between deprotection and coupling steps to remove residual deprotecting agent to minimize peptide impurities. Residual deprotecting agent can, for example, prematurely remove a protecting group (e.g., an Fmoc protecting group) from an amino acid to be coupled to the deprotected amino acid. This can result in undesirable insertion of an additional amino acid into the growing peptide chain. Residual deprotecting agent can also reduce amino acid activity by reacting with the amino acid, which can result in deletions in the peptide chain. The result is the generation of both insertions and deletions of the next amino acid which can lead to extremely difficult to separate impurities. For this reason, thorough washing (e.g., multiple washing steps) after any deprotection step is typically employed to prevent these potentially difficult to separate impurities.

Multiple washing steps can improve purity of shorter peptides. Even with multiple washing steps, however, the formation of impurities remains problematic, particularly as the number of amino acids in the peptide chain increases.

In addition, as compared to solution-based phase synthesis, SPPS can result in significant waste production from successive washing steps between each deprotection and coupling steps. Historically, this included about five (5) washes between each step, resulting in about 80-90% of the total waste being generated from washing. Chan, W. C., & White, P. D., Fmoc solid phase peptide synthesis: A practical approach. New York: Oxford University Press (2000).

There is accordingly a need for a solid phase peptide synthesis process and system that can provide peptide sequence purity, including improved purity of longer peptide sequences having, for example, up to 50, 100, or more, amino acid derived units; improve process efficiencies; and/or minimize or eliminate post-deprotection washing, which may reduce amounts of solvent required in SPPS processes and associated material costs, solvent disposal issues, etc.

### SUMMARY

The present disclosure relates to processes for deprotecting a protected amino acid (e.g., as a step in solid phase peptide synthesis). Deprotecting a protected amino acid (the deprotection reaction) removes a protecting group of the protected amino acid (deprotects the amino acid) to prepare the amino acid for a coupling reaction with a second amino acid.

Generally, the deprotection processes of the present disclosure use an inert gas to remove (flush, purge, vent, displace, replace, etc.) volatized deprotecting agent from a reaction vessel. In various embodiments, the deprotection processes of the present disclosure include a step of directing an inert gas through a portion of the interior of a reaction vessel including volatized deprotecting agent (e.g., directing an inert gas through the headspace of the reaction vessel including volatized deprotecting agent) to remove (flush, purge, vent, displace, replace, etc.) volatized deprotecting agent from the interior of the reaction vessel.

In a first embodiment, the process for deprotecting a protected amino acid (e.g., during solid phase peptide synthesis) includes the step of heating a protected amino acid and a deprotecting agent in a reaction vessel (e.g., in a lower interior portion of the reaction vessel) during a deprotection reaction. The deprotecting agent may generally have a lower boiling point relative to the temperature used to heat the protected amino acid and deprotecting agent and/or relative to the boiling points of solvents that may be present such as dimethylformamide (DMF) and N-methylpyrrolidinone (NMP). Accordingly, the deprotecting agent volatizes or evaporates during the heating step of the deprotection process (e.g., the deprotecting agent volatizes or evaporates into an upper interior portion of the reaction vessel).

In the first embodiment, the process for deprotecting a protected amino acid further includes the step of directing (e.g., continuously and/or intermittently directing) an inert gas through the interior of the reaction vessel during the heating step to remove (purge, flush, vent, displace, replace, etc.) volatized deprotecting agent from the interior of the reaction vessel. More specifically, in the first embodiment, the process may include directing (introducing, supplying, etc.) inert gas into the upper interior portion (e.g., headspace) of the reaction vessel including volatized deprotecting agent through a first opening located in an upper portion of the reaction vessel; and venting (flushing) the inert gas and evaporated deprotecting agent from the upper interior portion (e.g., from the headspace) of the reaction vessel through a second opening also located in an upper portion of the reaction vessel. Accordingly, in the first embodiment, the process may include directing inert gas into the upper interior portion of the reaction vessel through a first opening located in an upper portion of the reaction vessel, through the upper interior portion of the reaction vessel (e.g., through the headspace) including evaporated deprotecting agent and out of the upper interior portion (e.g., out of the headspace) of the reaction vessel through a second opening also located in the upper portion of the reaction vessel to remove (purge, flush, vent, displace, remove, etc.) volatized deprotecting agent from the upper interior portion of the reaction vessel (e.g., from the headspace of the reaction vessel).

In a second embodiment, the process for deprotecting a protected amino acid (e.g., during solid phase peptide synthesis) includes the step of removing a protecting group of a protected amino acid with a deprotecting composition, wherein the deprotecting composition includes a deprotecting agent in an amount of about 5 vol % or less, based on the total volume of the deprotecting composition. For example, the deprotecting composition may include the deprotecting agent in an amount from about 2 vol % to about 5 vol %, for example from about 2 to about 4.5 vol %, for example from about 3 to about 4.5 vol %, and as another example from about 3.5 to about 4.5 vol %, based on the total volume of the deprotecting composition.

In the second embodiment, the protected amino acid and the deprotecting composition are present in a lower interior portion of a reaction vessel. The deprotecting agent may generally have a lower boiling point relative to the deprotection reaction temperature and/or the temperature of the reaction vessel during deprotection and/or a lower boiling point relative to the boiling point of solvent(s) that may be present in the reaction vessel (e.g., solvents present in the deprotecting composition), such as dimethylformamide (DMF) and N-methylpyrrolidinone (NMP). Accordingly, at least a portion of the deprotecting agent evaporates (volatizes) into an upper interior portion of the reaction vessel during the removing step.

The process of the second embodiment may also include a step of directing (e.g., continuously and/or intermittently directing) an inert gas through the interior of the reaction vessel during the step of removing the protecting group to remove (flush, vent, purge, displace, replace, etc.) evaporated (volatized) deprotecting agent from the upper interior portion of the reaction vessel.

In the second embodiment, the directing step may include directing (introducing, supplying, etc.) inert gas into the upper interior portion (e.g., the headspace) of the reaction vessel including evaporated deprotecting agent; and venting (flushing) the inert gas and evaporated deprotecting agent from the upper interior portion (e.g., headspace) of the reaction vessel. More specifically, the directing step may include directing the inert gas into the upper interior portion (e.g., the headspace) of the reaction vessel through a first opening located in an upper portion of the reaction vessel and through the upper interior portion (e.g., the headspace) of the reaction vessel including evaporated deprotecting agent; and venting (flushing) the inert gas and evaporated deprotecting agent from the upper interior portion (e.g., from the headspace) of the reaction vessel through a second opening also located in an upper portion of the reaction vessel.

In the second embodiment, as another example, the directing step may include directing (introducing, supplying, etc.) inert gas into the lower interior portion of the reaction vessel; and venting (flushing) the inert gas and evaporated deprotecting agent from the upper interior portion (e.g., headspace) of the reaction vessel. More specifically, the directing step may include directing the inert gas into the lower interior portion of the reaction vessel through an opening located in a lower portion of the reaction vessel and upwardly from the lower interior portion of the reaction vessel (e.g., upwardly through reactants in the lower interior portion of the reaction vessel) through the upper interior portion (e.g., the headspace) of the reaction vessel including evaporated deprotecting agent; and venting (flushing) the inert gas and evaporated deprotecting agent from the upper interior portion (e.g., the headspace) of the reaction vessel through another opening located in an upper portion of the reaction vessel.

In the second embodiment, as yet another example, the directing step may include directing (introducing, supplying, etc.) inert gas into both the upper interior portion (e.g., the headspace) of the reaction vessel including evaporated deprotecting agent and into the lower interior portion of the reaction vessel; and venting (flushing) the inert gas and evaporated deprotecting agent from the upper interior portion (e.g., the headspace) of the reaction vessel. More specifically, the directing step may include directing inert gas into the upper interior portion (e.g., the headspace) of the reaction vessel through a first opening located in an upper portion of the reaction vessel and through the upper interior portion (e.g., the headspace) of the reaction vessel including evaporated deprotecting agent, and also directing inert gas into the lower interior portion of the reaction vessel through a second opening located in a lower portion of the reaction vessel and upwardly from the lower interior portion of the reaction vessel (e.g., upwardly through reactants in the lower interior portion of the reaction vessel) through the upper interior portion (e.g., the headspace) of the reaction vessel including evaporated deprotecting agent; and venting (flushing) the inert gas and evaporated deprotecting agent from the upper interior portion (e.g., the headspace) of the reaction vessel through a third opening located in an upper portion of the reaction vessel.

The second embodiment can include heating the protected amino acid and the deprotecting composition during the step of removing the protecting group from the protected amino acid, wherein at least a portion of the deprotecting agent evaporates into the upper interior portion of the reaction vessel during the heating step; and directing the inert gas through the interior of the reaction vessel to remove (vent, flush, etc.) inert gas and evaporated deprotecting agent from the upper interior portion of the reaction vessel during the heating step. Alternatively, in the second embodiment, the step of removing the protecting group may be conducted at room temperature.

The various embodiments of this disclosure can be alike, except for variations noted and variations that will be apparent to those of ordinary skill in the art. The present invention may, however, be embodied in many different forms and should not be construed as limited to the embodiments set forth herein. For example, features disclosed as part of one embodiment can be used in the context of another embodiment to yield a further embodiment. In the following, some of the embodiments may be named sequentially, but the sequence does not relate to relative preference.

When used, the heating step of any of the embodiments of the deprotection processes disclosed herein may be conducted, for example, at a temperature from about 40° C. to about 120° C., as another example about 50° C. to about 120° C., and as another example about 70° C. to about 120° C. The heating step of any of the embodiments of the deprotection processes disclosed herein may be conducted using microwave radiation.

The present disclosure also relates to processes for solid phase peptide synthesis (SPPS). The SPPS processes include the step of deprotecting a first protected amino acid to provide a deprotected amino acid in accordance with any of the embodiments described herein (e.g., the first embodiment and/or the second embodiment of the deprotecting step); and coupling a second amino acid to the deprotected amino acid to form a peptide from the first and second amino acids.

In some embodiments (e.g., the second embodiment), the SPPS processes may not include a washing step after the deprotecting step and before the coupling step. In some embodiments (e.g., the second embodiment), the SPPS processes may include a washing step after the deprotection step and before the coupling step using a washing composition (e.g., solvent) in an amount that is less than the total volume of the deprotecting composition. For example, the washing step may include washing using a washing composition in an amount that is less than ½ of the total volume of the deprotecting composition, and as another example washing using a washing composition in an amount that is less than ⅓ of the total volume of the deprotecting composition.

The present disclosure also relates to a system for solid phase peptide synthesis.

The present disclosure can provide various unexpected benefits. The processes of the present disclosure can provide good (e.g., improved) peptide purity, even for longer peptides including up to 50, 100, or more amino acids, relative to peptide purity achieved using SPPS processes without the use of an inert gas to remove (flush, purge, vent, etc.) evaporated deprotecting agent from the reaction vessel as described herein. The processes of the present disclosures may also allow the use of more reactive deprotecting agents with lower boiling points (e.g., pyrrolidine) at higher temperatures, which can accelerate reaction times, while minimizing adverse effects associated with using a low boiling point, readily volatized reactant.

Thus, the processes can improve peptide purity, even for long peptide chains, and even using pyrrolidine as the deprotecting agent, despite significant evaporating observed due to its lower boiling point. The processes can also provide benefits of faster deprotection rates.

The processes of the present disclosure may also provide effective SPPS results (for example, as measured by peptide purity) using relatively small amounts of a deprotecting agent (e.g., about 5 vol % or less, relative to the total volume of deprotecting composition), and/or without requiring a washing step after deprotection and before coupling, and/or with a washing step after deprotection and before coupling using reduced amounts of solvent (e.g., using solvent in an amount that is less than the total volume of the deprotecting composition). This can also provide benefits such as improved process efficiencies, energy savings, reduced amounts of solvent required in the SPPS process, reduced material costs, reduced solvent disposal issues, etc.

The foregoing illustrative summary, as well as other exemplary examples, objectives and/or advantages of the invention, and the manner in which the same are accomplished, are further explained within the following detailed description and its accompanying drawings. The foregoing summary provides a few brief examples and is not exhaustive, and the present invention is not limited to the foregoing examples.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. The present invention may be embodied in many different forms and should not be construed as limited to the examples depicted in the drawings.

FIG. 1A is a cross-sectional view of an exemplary reaction vessel and schematically depicts a process of deprotecting a protected amino acid in accordance with embodiments of the present disclosure;

FIG. 1B is a cross-sectional view of another exemplary reaction vessel and schematically depicts a process of deprotecting a protected amino acid in accordance with other embodiments of the present disclosure;

FIG. 2A is a schematic flow diagram depicting selected portions of an exemplary peptide synthesis system in accordance with embodiments of the present disclosure;

FIG. 2B is a schematic flow diagram depicting selected portions of an exemplary peptide synthesis system in accordance with other embodiments of the present disclosure;

FIG. 3A is an ultra performance liquid chromatography (also referred to herein as “UPLC”) chromatograph of thymosin synthesized by solid phase peptide synthesis utilizing headspace purging (e.g., continuous headspace cleaning) during deprotection step(s) as described in Example 1 in accordance with embodiments of the present disclosure;

FIG. 3B is an UPLC chromatograph of thymosin synthesized using solid phase peptide synthesis without headspace purging (e.g., without continuous headspace cleaning) during deprotection step(s) as described in Comparative Example 1;

FIG. 4A is an UPLC chromatograph of proinsulin synthesized using solid phase peptide synthesis with headspace purging (e.g., continuous headspace cleaning) during deprotection step(s) as described in Example 2 in accordance with embodiments of the present disclosure;

FIG. 4B is a mass spectrum of proinsulin synthesized using solid phase peptide synthesis with headspace purging (e.g., continuous headspace cleaning) during deprotection step(s) as described in Example 2 in accordance with embodiments of the present disclosure;

FIG. 5A is an UPLC chromatograph of HIV-1 protease synthesized using solid phase peptide synthesis with headspace purging (e.g., continuous headspace cleaning) during deprotection step(s) as described in Example 3 in accordance with embodiments of the present disclosure;

FIG. 5B is a mass spectrum of HIV-1 protease synthesized using solid phase peptide synthesis with headspace purging (e.g., continuous headspace cleaning) during deprotection step(s) as described in Example 3 in accordance with embodiments of the present disclosure;

FIG. 6A is an UPLC chromatograph of Barstar synthesized using solid phase peptide synthesis with headspace purging (e.g., continuous headspace cleaning) during deprotection step(s) as described in Example 4 in accordance with embodiments of the present disclosure;

FIG. 6B is a mass spectrum of Barstar synthesized using solid phase peptide synthesis with headspace purging (e.g., continuous headspace cleaning) during deprotection step(s) as described in Example 4 in accordance with embodiments of the present disclosure;

FIG. 7A is an UPLC chromatograph of MDM2 synthesized using solid phase peptide synthesis with headspace purging (e.g., continuous headspace cleaning) during deprotection step(s) as described in Example 5 in accordance with embodiments of the present disclosure; and

FIG. 7B is a mass spectrum of MDM2 synthesized using solid phase peptide synthesis with headspace purging (e.g., continuous headspace cleaning) during deprotection step(s) as described in Example 5 in accordance with embodiments of the present disclosure.

### DETAILED DESCRIPTION

The above and other aspects, features, and advantages of the present invention will become apparent from the detailed description of the following embodiments. It should be understood that the present invention is not limited to the following embodiments and may be embodied in different ways by those skilled in the art without departing from the scope of the present invention. Rather, the embodiments are provided for complete disclosure and to provide thorough understanding of the present invention by those skilled in the art. The scope of the present invention should be defined only by the appended claims.

Embodiments of the present disclosure relate to processes and systems for deprotecting a protected amino acid (e.g., as a step in solid phase peptide synthesis). In exemplary embodiments, the processes and systems are batch-based processes and systems.

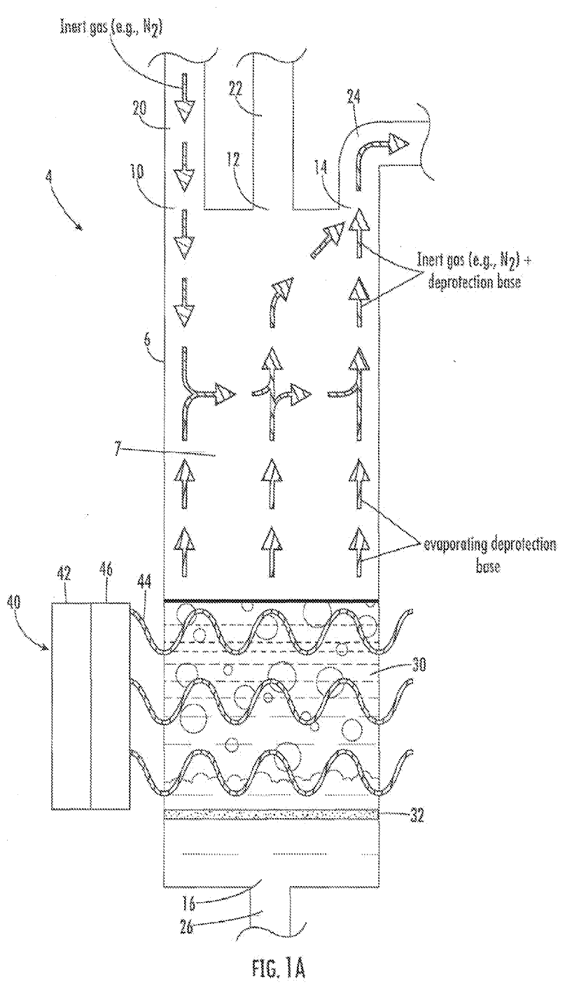

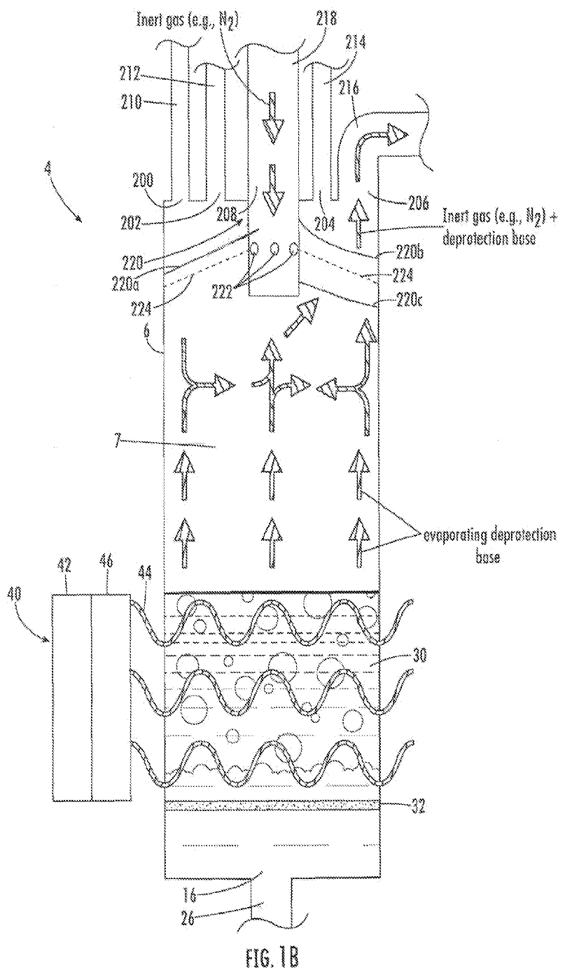

FIG. 1A is a schematic cross-sectional view of a reaction vessel suitable for use in the amino acid deprotecting and peptide synthesis processes and systems in accordance with embodiments of the present disclosure. FIG. 1A also schematically depicts a process according to an embodiment of the present disclosure for deprotecting a protected amino acid.

FIG. 1B is a schematic cross-sectional view of another reaction vessel suitable for use in the amino acid deprotecting and peptide synthesis processes and systems in accordance with embodiments of the present disclosure. FIG. 1B also schematically depicts a process according to an embodiment of the present disclosure for deprotecting a protected amino acid.

Except where indicated otherwise, elements illustrated in FIG. 1A will carry the same reference numerals as in the other Figures, including FIG. 1B.

As shown in FIGS. 1A and 1B, a reaction vessel 4 includes at least one side wall 6 extending around an interior 7 (e.g., cavity) of reaction vessel 4 (the interior 7 of the reaction vessel also referred to herein as an outer interior space). The specific size and shape of reaction vessel 4 are not limited. Reaction vessels suitable for use in solid phase peptide synthesis are well known in the art and are commercially available.

The size (interior volume) of the reaction vessel is not limited. Exemplary reaction vessel sizes can range from less than 1 liter up to 40 liters, or more, for example, 10 ml, 30 ml, 125 ml, 1 liter, 3 liters, 5 liters, 8 liters, 10 liters, 15 liters, etc. up to 40 liters, or more, without limitation.

Reaction vessel 4 further includes one or more openings. As a non-limiting example, FIG. 1A depicts openings 10, 12, and 14 located in an upper portion (e.g., in a top wall) of reaction vessel 4, and an opening 16 located in a lower portion (e.g., a bottom wall) of reaction vessel 4. As another non-limiting example, FIG. 1B depicts openings 200, 202, 204, 206, and 208 located in an upper portion (e.g., in a top wall) of reaction vessel 4, and an opening 16 located in a lower portion (e.g., a bottom wall) of reaction vessel 4. The openings (e.g., inlets, outlets, ports, etc.) allow the introduction and/or removal of fluids (e.g., gases and/or liquids) and/or solids, such as reactants, solvents, gases, products (peptides), byproducts, excess (residual) reactants, and the like as discussed in more detail herein.

The skilled artisan will understand that the reaction vessel 4 is not limited to the number and/or locations of openings depicted in FIGS. 1A and 1B and that other reaction vessel designs and configurations having fewer or more openings and/or different locations thereof can be used (e.g., the reaction vessel may include fewer or more openings located in a top wall and/or a bottom wall and/or a side wall, etc.).

Fluids and/or solids can be introduced (e.g., moved, transported, directed, flushed, purged, evacuated, vented, etc.) into and out of reaction vessel 4 via one or more flow paths (e.g., lines, passageways, tubes, manifolds, etc.), such as flow paths 20, 22, and 24 in fluid communication with openings 10, 12, and 14, respectively, and flow path 26 in fluid communication with opening 16, as depicted in FIG. 1A.

Other non-limiting examples of flow paths are depicted in FIG. 1B as flow paths 210, 212, 214, 216, and 218 in fluid communication with openings 200, 202, 204, 206, and 208, respectively, and flow path 26 in fluid communication with opening 16.

In some embodiments, reaction vessel 4 can include at least one spray head (e.g., spray nozzle) or equivalent structure located in the interior of the reaction vessel for adding (e.g., directing, supplying, spraying, etc.) fluid (e.g., solvent, reactant, and/or inert gas) into the reaction vessel. The spray head can be a part (e.g., component, element, etc.) that is separate from the reaction vessel (e.g., can be installed in and removed from the reaction vessel) or can be an integrated part of the reaction vessel.

As a non-limiting example, FIG. 1B schematically depicts an embodiment including a spray head 220 located in the interior space 7 (also referred to herein as the outer interior space) of reaction vessel 4. Spray head 220 includes at least one side wall 220a positioned in the interior space 7 (also referred to as the outer interior space) of reaction vessel 4 and extending around an inner interior space. Spray head 220 includes a first portion (end) 220b proximate opening 208 in fluid communication with opening 208 (and flow path 218) and a second portion (end) 220c distal opening 208. A plurality of holes 222 (e.g., ports, etc.) extend at least partially around the inner interior space and can be, for example, defined in side wall 220a. The inner interior space and the outer interior space are in fluid communication with one another by way of holes of the plurality of holes.

Spray head 220 is configured so that fluid (such as inert gas, solvent, etc. as discussed herein) directed into the inner interior space of spray head 220 via flow path 218 and opening 208 passes (e.g., is sprayed, directed, supplied, etc.) through holes (e.g., through one, more than one, a majority, or all) of the plurality of holes 222 into the outer interior space 7 of reaction vessel 4. In some embodiments, spray head 220 can be configured so that fluid exiting at least one or more holes of the plurality of holes 222 is directed (sprayed) towards side wall 6 of the reaction vessel. An exemplary spray pattern is schematically depicted in FIG. 1B by dashed lines 224 (e.g., in an approximate manner), in which fluid exiting holes of the plurality of holes 222 is directed at a generally downward angle towards side wall 6.

The present disclosure is not limited with respect to a specific spray head structure, location in the reaction vessel, spray pattern and/or direction (angle) of fluid exiting holes of the spray head, such as illustrated in FIG. 1B, and other spray head structures, locations, spray patterns, and/or spray angles, etc., can be used. Spray heads suitable for use in SPPS processes and systems are known in the art and can be used in the present disclosure.

The present disclosure is not limited to a specific number and/or locations of openings and flow paths and the reaction vessel can accordingly have one, two, three, four, or more openings and associated flow paths as appropriate. Further, any series of flow paths and associated valves that serve to direct, allow, and/or block (e.g., close, restrict, etc.) the flow of fluids and/or solids can be used.

In the deprotecting processes of the present disclosure, reactants designated at 30 are present in a lower portion of reaction vessel 4. For example, the reactants can rest on a filter 32 located in the lower portion of reaction vessel 4. Filter 32 can also prevent a solid support (e.g., solid resin support) such as discussed herein from entering flow path 26.

Reactants 30 include a protected amino acid, i.e., an amino acid including at least one protecting group attached to a functional group, such as a terminal amine group, to protect from unwanted reactions of the functional group.

Reactants 30 also include a deprotecting agent. The deprotecting agent reacts with the protected amino acid to remove the protecting group and to make the previously protected functional group (e.g., a terminal amine group) available for reaction (e.g., with one, two, or more, successive amino acid to form a peptide chain).

Deprotecting agents used in SPPS processes are typically liquid at room temperature. Thus, the deprotecting agent is typically added to and/or present in the reaction vessel as a part of a deprotecting composition including the deprotecting agent (the amount of deprotecting agent is greater than zero volume % based on the total volume of the deprotecting composition) and a suitable solvent.

The deprotecting agent can be an organic base. Examples of an organic base suitable for use as a deprotecting agent include without limitation piperidine and/or pyrrolidine. Other organic bases that provide the deprotection function without otherwise interfering with the other steps in the process, the growing peptide chain, or the system, can be appropriate as well.

Examples of solvents that can be part of the deprotecting composition may include without limitation dimethylformamide (DMF), dimethylacetamide (DMA), N-methylpyrrolidinone (NMP), green solvents and/or non-reprotoxin solvents, and the like, and combinations and/or mixtures thereof. Examples of green and/or non-reprotoxin solvents may include without limitation N-formylmorpholine (NFM), N-butylpyrrolidinone (NBP), alkoxybenzene-based solvents (e.g., anisole, dimethoxybenzene-based solvents such as 1,3-dimethoxoybenzene, etc.), and the like, and combinations and/or mixtures thereof.

In embodiments of the present disclosure (e.g., the second embodiment), the deprotecting composition may include the deprotecting agent in an amount of about 5 volume % (vol %) or less, based on the total volume (100% volume) of the deprotecting composition (the deprotecting agent is present, i.e., the amount of deprotecting agent is greater than zero). In some embodiments (e.g., the second embodiment), the deprotecting composition may include the deprotecting agent in an amount from about 1 vol % to about 5 vol %, for example from about 2 vol % to about 5 vol %, for example from about 2 to about 4.5 vol %, for example from about 3 to about 4.5 vol %, and as another example from about 3.5 to about 4.5 vol %, based on the total volume of the deprotecting composition. In some embodiments (e.g., the second embodiment), the deprotecting composition may include the deprotecting agent in an amount of greater than zero to about 4.5 vol %, based on the total volume of the deprotecting composition. In some embodiments (e.g., the second embodiment), the deprotecting composition may include the deprotecting agent in an amount of greater than zero to about 4 vol %, based on the total volume of the deprotecting composition. In some embodiments (e.g., the second embodiment), the deprotecting composition may include the deprotecting agent in an amount of greater than zero to about 3 vol %, based on the total volume of the deprotecting composition. The amount of deprotecting agent (e.g., in the second embodiment) may be any value within the ranges described herein, including end points (e.g., any value within a range of greater than zero to about 5 vol %) and all subranges within the range are also disclosed.

The concentration of deprotecting agent in the deprotecting composition, however, is not so limited and may vary. For example, in some embodiments of the present disclosure (e.g., the first embodiment), the deprotecting composition may include the deprotecting agent in an amount of greater than about 5 vol %, for example, about 20 vol % or more, based on the total volume (100% volume) of the deprotecting composition. In some embodiments (e.g., the first embodiment), the deprotecting composition may include the deprotecting agent in an amount from about 20 vol % to 100 vol %, for example from about 20 to about 50 vol %, for example from about 20 vol % to about 40 vol %, and as another example from about 20 to about 35 vol %, based on the total volume of the deprotecting composition. The amount of deprotecting agent (e.g., in the first embodiment) may be any value within the ranges described herein, including end points (e.g., any value within a range of greater than about 5 vol % to 100 vol %) and all subranges within the range are also disclosed.

The skilled artisan will understand the meaning of the term amino acid. As used herein, the term amino acid in its broadest sense refers to organic compounds that contain both amine and carboxylic acid functional groups, and in some instances also a side chain. The skilled artisan will also understand that amino acids include natural amino acids (proteinogenic amino acids) and/or non-proteinogenic amino acids, and will also understand the single letter designations used to identify the same.

The term peptide will also be understood by the skilled artisan. As used herein, the term peptide has its ordinary meaning in the art and may refer to amides derived from two or more amino acids (the same or different) by bonding the carbonyl carbon of one amino acid to the nitrogen atom of another amino acid.

Suitable protecting groups for use in the processes of the present disclosure are well-known in the art. An example of a protecting group suitable for protection of amine or N-terminus includes without limitation a fluorenylmethyloxycarbonyl (Fmoc) protecting group. See, for example, Chan and White, Fmoc solid phase peptide synthesis, a practical approach, Oxford University Press (2000).

The amino acid can also include side-chain protecting groups. Examples of side-chain protecting groups can include without limitation trityl, t-butyl, and/or 2,2,4,6,7-pentamethyldihydrobenzofuran-5-sulfonyl (Pbf) protecting groups, and the like. When the desired peptide chain length has been obtained, the side-chain protecting groups can be removed.

The protected amino acid can be directly or indirectly attached to a solid support as known in the art. For example, the carboxy terminus of the protected amino acid can be attached to the solid support via a suitable linker. As another example, the carboxy terminus of the protected amino acid can be indirectly attached to the solid support, for example, can be linked to another amino acid (or to a peptide chain) that is in turn linked to the solid support.

Solid supports known in the art may be used in the processes of the present disclosure. Non-limiting examples of solid support materials include polystyrene (e.g., in resin form such as microporous polystyrene resin, mesoporous polystyrene resin, macroporous polystyrene resin), glass, polysaccharides (e.g., cellulose, agarose), polyacrylamide resins, polyethylene glycol, and/or copolymer resins (e.g., comprising polyethylene glycol, polystyrene, etc.). In exemplary embodiments, the solid support is a solid phase resin.

The solid support may have any suitable form. For example, the solid support can be in the form of beads, particles, fibers, and/or in any other suitable form.

The skilled artisan will understand how link an amino acid to a solid support (e.g., a solid phase resin). Accordingly, a detailed discussion of methods known in the art for linking an amino acid (and/or a peptide including two or more amino acids) to a solid support (e.g., a solid phase resin) is not provided.

The deprotecting agent and protected amino acid (attached directly or indirectly to a suitable solid support such as a solid phase resin as discussed herein) can be mixed (combined) with a suitable solvent(s) as known in the art and can be in the form of a suspension (slurry) in the reaction vessel.

As noted herein, the deprotection reaction removes a protecting group from a protected group (e.g., protected functional group) of a protected amino acid (also generally referred to as deprotecting the amino acid).

In some embodiments (e.g., the second embodiment), the deprotecting step can be conducted without heat (e.g., can be conducted at room temperature), so long as the deprotecting conditions (type of base, time, etc.) are selected to promote evaporation of the deprotecting agent into the headspace of the reaction vessel.

More typically, in some embodiments (e.g., the first and/or second embodiments), the deprotecting process may include the step of heating the protected amino acid and/or the deprotecting agent (e.g., heating the protected amino acid and/or the deprotecting agent before delivery into the reaction vessel 4 and/or heating the protected amino acid and/or the deprotecting agent in the reaction vessel 4 before and/or during the deprotection reaction). As used herein, reference to a deprotecting agent may include a deprotecting agent per se and/or a deprotecting composition including a deprotecting agent. Thus, as used herein, reference to heating a deprotecting agent may include heating a deprotecting agent per se and/or heating a deprotecting composition including a deprotecting agent.

Heating during solid phase peptide synthesis can be useful, for example, to accelerate the rate of deprotection and thereby reduce the amount of time required for peptide synthesis.

Heating temperatures (e.g., heating temperatures of the protected amino acid and/or the deprotecting agent before delivery into the reaction vessel and/or in the reaction vessel before and/or during deprotection) can vary. In some embodiments, the heating step can be conducted at a temperature from about 40° C. to about 120° C., as another example from about 50° C. to about 120° C., as another example from about 70° C. to about 120° C., as another example from about 80° C. to about 120° C., as another example from about 80° C. to about 110° C., and as another example from about 90° C. to about 110° C., without limitation. In certain embodiments, the heating step can be conducted at a temperature from about 70° C. to about 120° C., for example from about 90° C. to about 120° C., and as another example from about 90° C. to about 110° C., without limitation. The temperature may be any value within the ranges described herein, including end points (e.g., any value within a range of from about 40° C. to about 120° C.) and all subranges within the range are also disclosed.

FIGS. 1A and 1B schematically depict a heating step wherein a heat source 40 heats reaction vessel 4 and reactants 30. In certain embodiments, heat source 40 includes a microwave source 42 positioned to direct microwave radiation 44 through a waveguide 46 attached to a microwave cavity (not illustrated) containing reaction vessel 4. Microwave power may be regulated as known in the art to provide reaction temperatures and/or reaction times (e.g., without limitation, to provide deprotection temperatures as described herein and to provide deprotection reaction times ranging from about 10 sec to about 15 minutes, as another non-limiting example from about 40 sec to about 8 minutes).

In embodiments utilizing microwave energy to heat the reactants, reaction vessel 4 can be formed of a material that is transparent to microwave radiation, such as but not limited to glass, Teflon, and/or polypropylene.

Microwave sources are well known in the art and can include, for example, magnetrons, klystrons, and/or solid-state diodes. Microwave sources, waveguides and microwave cavities suitable for solid phase peptide synthesis processes and systems are well known in the art and also are commercially available (e.g., systems commercially available from CEM Corporation such as discussed herein). Accordingly, the skilled artisan will understand how to use the same in solid phase peptide synthesis processes and systems without undue experimentation.

The present disclosure, however, is not limited to the use of microwave sources as the heat source, and other types of heat sources known in the art for solid phase peptide synthesis can used.

Despite the benefits of heating, elevated temperatures during the deprotecting step can present various challenges for peptide synthesis (including, but not limited to, the synthesis of longer peptides).

For example, organic amines used in deprotection reactions can have relatively low boiling points, as compared to the boiling point of a solvent used in a deprotection reaction and/or the temperature of the deprotecting step. Piperidine has a boiling point of about 106° C. and pyrrolidine has a boiling point of about 87° C. In contrast, the solvent dimethylformamide (DMF) has a boiling point of about 153° C. and the solvent N-methylpyrrolidinone (NMP) has a boiling point of about 200° C. Also in contrast, as noted herein, deprotecting reactions can be conducted at elevated temperatures, for example up to about 120° C., for example about 90° C. to about 120° C., and as another example about 90° C. to about 110° C., without limitation.

A reaction vessel can exhibit a temperature continuum during processing, wherein an upper portion thereof can be at a lower temperature than lower portions. Because the deprotecting agent can have a boiling point lower than the boiling point of other agents such as solvents and/or lower than reaction temperatures, the deprotecting agent can volatize (evaporate) into the upper portion of the reaction vessel (e.g., the headspace) and condense on upper portions of a side wall(s) and/or on a top wall of the reaction vessel. The rate/amount of volatilization (evaporation) can also increase, for example, when the reactants are bubbled during deprotection to help mix the reactants.

Volatilization of a deprotecting agent can be especially problematic using pyrrolidine. Pyrrolidine would be desirable as a deprotecting agent because pyrrolidine can provide faster deprotection than piperidine. As a 5-membered ring (versus a 6-membered piperidine ring), the carbon atoms of pyrrolidine are bent back more from the nitrogen atom, which facilitates an easier attack for deprotection. Because pyrrolidine has a lower boiling point than piperidine, however, significant evaporation followed by condensation can occur during deprotection processes, thereby limiting its use, including for example in the synthesis of long peptides.

In processes of the present disclosure, the heating step volatizes (evaporates) the deprotecting agent (e.g., pyrrolidine) from the lower portion of reaction vessel 4 upwardly into the upper portion (e.g., into the headspace above reactants 30) of reaction vessel 4.

Residual deprotecting agent remaining in a reaction vessel (e.g., residual deprotecting agent condensed on upper portions of a side wall(s) and/or on a top wall of the reaction vessel) during subsequent solid phase peptide synthesis steps (such as coupling steps) can be problematic. Residual deprotecting agent can, for example, prematurely remove a protecting group from an amino acid to be coupled to the already deprotected amino acid. This can result in undesirable insertions into the peptide chain. Residual deprotecting agent can also reduce activated amino acid by reacting with the amino acid, which can result in deletions in the peptide chain. Accordingly, typically SPPS processes have required multiple washing steps after deprotection and before coupling as discussed herein.

Conventionally, multiple washing steps are used to help remove residual deprotecting agent to minimize or prevent participation thereof in subsequent solid phase peptide synthesis steps (e.g., a coupling step). As peptide length increases, however, multiple washing steps are less effective in preventing unwanted reactions and reducing impurities, and it can be difficult to synthesize longer peptides with purities acceptable for downstream applications.

In contrast to conventional processes, the processes of the present disclosure can facilitate the production of longer peptides having acceptable purity levels for downstream applications. The present disclosure is not limited, however, to the production of longer peptides and may generally facilitate the production of peptides having acceptable purity levels for downstream application regardless of peptide length. Also in contrast to conventional processes, in some embodiments (e.g., the second embodiment), the processes of the present disclosure can help eliminate washing step(s) between deprotecting and coupling steps and/or reduce the amount of solvent required for a washing step(s) between deprotecting and coupling steps of a SPPS process and/or may facilitate the use of smaller amounts of deprotecting agent (base), as compared to conventional SPPS processes. Again without limitation, in some embodiments (e.g., the first embodiment and/or the second embodiment of the deprotecting step), it is currently believed that peptide purity and/or eliminated washing steps and/or reduced solvent amounts for washing steps and/or reduced amounts of deprotecting base may be facilitated by the step of directing (e.g., continuously and/or intermittently directing) an inert gas through a portion of the interior of a reaction vessel including evaporated deprotecting agent (e.g., directing the inert gas through the upper interior portion (through the headspace above the reactants) of the reaction vessel including evaporated deprotecting agent) during the deprotection step to remove (e.g., flush, vent, discharge, displace, replace, purge, etc.) evaporated (volatized) deprotecting agent from the interior of the reaction vessel (e.g., from the reaction vessel headspace).

More specifically, in various embodiments (e.g., the first and/or second embodiments) described herein, the deprotecting step can include directing (introducing, supplying, etc.) inert gas into the interior of the reaction vessel via one or more openings of the reaction vessel. For example, the inert gas may be directed into an upper interior portion and/or a lower interior portion of the reaction vessel via one or more openings (entry ports) located in an upper portion and/or a lower portion of the reaction vessel, respectively. The directing step may further include directing (moving, etc.) inert gas through the upper interior portion (e.g., through the headspace above the reactants) of the reaction vessel including evaporated deprotecting agent (and in some embodiments directing inert gas upwardly from the lower interior portion into/through the upper interior portion, or headspace, including evaporated deprotecting agent); and removing (e.g., flushing, venting, discharging, displacing, replacing, purging, etc.) evaporated (volatized) deprotecting agent from the upper interior portion (e.g., from the headspace above the reactants) of the reaction vessel through one or more other openings (exit ports) located in the upper portion of the reaction vessel.

In some embodiments, the directing step may include directing inert gas into an upper interior portion (e.g., the headspace above the reactants) of the reaction vessel including volatized deprotecting agent via a first opening located in an upper portion of the reaction vessel and through the upper interior portion (e.g., through the headspace above the reactants) of the reaction vessel including evaporated deprotecting agent to remove (e.g., flush, vent, discharge, displace, replace, purge, etc.) the inert gas and volatized deprotecting agent from the upper interior portion (e.g., from the headspace above the reactants) through a second opening also located in an upper portion of the reaction vessel. In some embodiments, the directing step may include directing inert gas into a lower interior portion of the reaction vessel via a first opening located in a lower portion of the reaction vessel and upwardly from the lower interior portion of the reaction vessel (e.g., upwardly through reactants in a lower interior portion of the reaction vessel) into/through the upper interior portion (e.g., through the headspace above the reactants) of the reaction vessel including evaporated deprotecting agent to remove (e.g., flush, vent, discharge, displace, replace, purge, etc.) evaporated (volatized) deprotecting agent from the upper interior portion (e.g., from the headspace above the reactants) through a second opening located in an upper portion of the reaction vessel. In some embodiments, the directing step may include directing inert gas into an upper interior portion (e.g., the headspace above the reactants) of the reaction vessel including volatized deprotecting agent via a first opening located in an upper portion of the reaction vessel and through the upper interior portion (e.g., through the headspace above the reactants) of the reaction vessel including evaporated deprotecting agent, and also directing inert gas into a lower interior portion of the reaction vessel through a second opening located in a lower portion of the reaction vessel, the inert gas optionally moving upwardly from the lower interior portion of the reaction vessel and into/through the headspace of the reaction vessel, to remove (e.g., flush, vent, discharge, displace, replace, purge, etc.) evaporated (volatized) deprotecting agent from the upper interior portion (e.g., from the headspace above the reactants) through a third opening located in an upper portion of the reaction vessel.

In some embodiments, the inert gas can be continuously directed through the reaction vessel as a continuous flow. In some embodiments, the inert gas can be directed through the reaction vessel as an intermittent (e.g., pulsed) flow.

In some embodiments, the inert gas directed into and/or moving through the reaction vessel (e.g., moving through the headspace of the reaction vessel including volatized deprotecting agent) may have a pressure of about 1 psi to about 25 psi. In some embodiments, the inert gas directed into and/or moving through the reaction vessel (e.g., moving through the headspace of the reaction vessel including volatized deprotecting agent) may have a pressure of about 1, 2, 3, 4, 5, 6, 7, 8, 9, 10, 11, 12, 13, 14, 15, 16, 17, 18, 19, 20, 21, 22, 23, 24, or 25 psi. In some embodiments, the inert gas directed into and/or moving through the reaction vessel may have a pressure in a range from about any of the foregoing pressure values to about any other of the foregoing pressure values. The pressure of the inert gas, including inert gas directed into and/or moving through an upper interior portion and/or a lower interior portion of the reaction vessel, may be any value within the ranges described herein, including end points (e.g., any value within a range of from about 1 to about 25 psi) and all subranges within the range are also disclosed. The inert gas may also be directed through the interior (e.g., through an upper interior portion, or headspace) of the reaction vessel during the deprotection step at a flow rate based on a time rate within which the inert gas substantially replaces (displaces) the headspace gas volume. More specifically, the inert gas flow rate may be an amount (volume) of inert gas that allows for (results in) substantial replacement (displacement) of the volume of gas in the headspace area of the reaction vessel with (by) the inert gas (e.g., that results in substantial replacement of the volume of volatized deprotecting agent in the headspace area of the reaction vessel with the inert gas) within a selected time period (time rate). For example, the inert gas flow rate may be an amount (volume) of inert gas that results in (allows for) the substantial replacement (displacement) of the volume of gas in the headspace area (e.g., the volume of volatized deprotecting agent in the headspace area) of the reaction vessel about every one (1) to twenty (20) seconds, for example, about every five (5) to ten (10) seconds. The skilled artisan will understand how to determine and calculate suitable inert gas flow rates to replace (displace) a volume of headspace gas (volatized deprotecting agent) in a reaction vessel within a time frame (time rate) without undue experimentation.

Although not wishing to be bound by any explanation or theory, it is currently believed that directing a source of inert gas into (through) the headspace above the reactants during deprotection can cause a large air exchange rate in the gas above the reactants (the headspace gas including the volatized deprotecting agent) such that the inert gas displaces the volatized deprotecting agent from the reaction vessel. This can reduce residence time of the volatized deprotecting agent in the reaction vessel and the volatized deprotecting agent can be more quickly removed with less condensation on the side and/or top walls of the vessel. This in turn can reduce the amount of residual deprotecting agent remaining in the reaction vessel after the deprotecting step is completed. The inert gas can also provide downward force on droplets (e.g., condensed deprotecting agent) on a side wall of reaction vessel 4 and can thereby blow the droplets toward reactants 30 in the lower portion of reaction vessel 4.

Further, in some embodiments (e.g., the second embodiment) in which the deprotecting composition includes a low amount of deprotecting agent (about or less than about 5 vol % deprotecting agent based on the total volume of deprotecting composition), the deprotecting agent (e.g., pyrrolidine) may be essentially completely removed from the reaction vessel upon completion of the deprotection step. For example, without being bound by any theory or explanation, in such embodiments, it is currently believed that the deprotecting agent may substantially completely evaporate from the deprotecting composition during a heating step and/or volatized deprotecting agent may be substantially completely removed from the headspace using inert gas flushing, each as described herein.

Also without being bound by any theory or explanation, in such embodiments, it is currently believed that any residual amount of deprotecting agent remaining after completion of the deprotection step is small enough to minimize issues associated with the presence of residual deprotecting agent in the next coupling step, even without a washing step after the deprotection step and/or with a washing step after the deprotection step using reduced amounts of washing liquid (e.g. solvent) also as described in more detail herein.

Because the amount of residual deprotecting agent can be reduced, in contrast to conventional approaches, the process of the present disclosure can facilitate the production of longer peptides having acceptable purity levels for downstream applications. For example, the present process can be useful for the production (e.g., batch SPPS synthesis) of longer peptides, such as but not limited to peptides including 20 or more amino acid derived units, for example 25 or more amino acid derived units, as another example 30 or more amino acid derived units, as another example 40 or more amino acid derived units, as another example 50 or more amino acid derived units, as another example 75 or more amino acid derived units, as another example 100 or more amino acid derived units, as another example 125 or more amino acid derived units, and as another example 150 or more amino acid derived units. In some embodiments, the process can be useful for the production (e.g., batch SPPS synthesis) of longer peptides, such as but not limited to peptides including 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more amino acid derived units. The present disclosure is not limited, however, to the production of peptides including 20 or more amino acid derived units and can be used for the production (e.g., batch SPPS synthesis) of peptides including fewer than 20 amino acid derived units (e.g., for peptides including two or more, for example two to 20, or more, amino acid derived units), wherein the peptides may have acceptable purity levels for downstream applications.

The inert gas can be nitrogen. The present disclosure is not limited to the use of nitrogen as the inert gas and other inert gases, such as the noble gases, with limited or no interference chemically with solid phase peptide synthesis reactions and with the solid phase peptide synthesis system can be used.

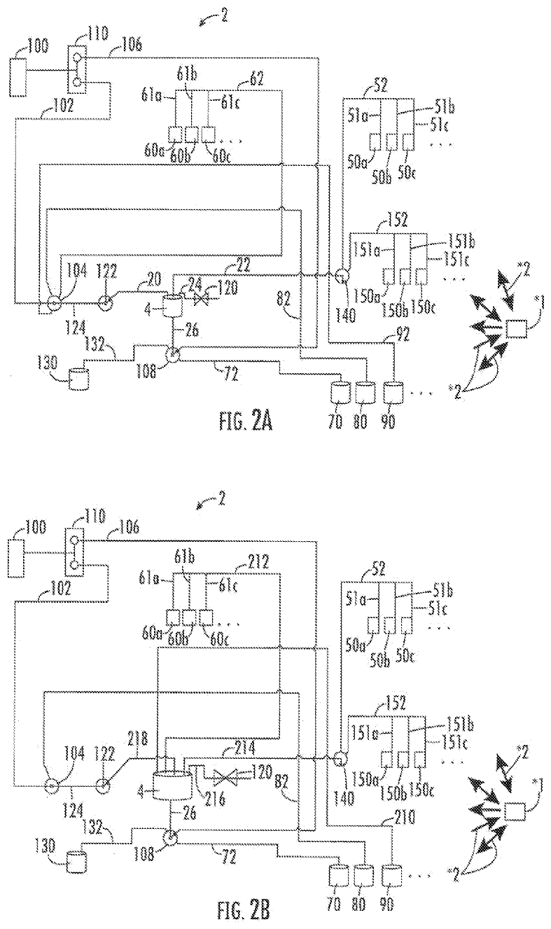

In certain embodiments, as depicted in FIG. 1A, the process can include providing pressurized inert gas from an inert gas source (such as inert gas source designated as 100 in FIG. 2A) through flow path 20 and directing the pressurized inert gas (e.g., generally downwardly directing the pressurized inert gas) into the upper portion of the interior of the reaction vessel 4 through opening 10 and through the upper interior portion of the reaction vessel 4 (e.g., through the headspace above reactants 30) including volatized (evaporated) deprotecting agent. As the pressurized inert gas flows through the upper interior portion (e.g., through the headspace above the reactants) of the reaction vessel 4 including volatized (evaporated) deprotecting agent, the inert gas purges (e.g., flushes, displaces, replaces, vents, discharges, etc.) volatized deprotecting agent out of the interior of the reaction vessel 4 through opening 14 into flow path 24. In this manner, the pressurized inert gas in effect displaces the volatized deprotecting gas from the headspace of the reaction vessel. This can reduce residence time and minimize condensation of the deprotecting agent on the walls of the reaction vessel.

Gas flow (movement) in reaction vessel 4, including upward flow of volatized deprotecting agent (e.g., pyrrolidine) from reactants 30 in the lower interior portion of reaction vessel 4 into the headspace above the reactants (e.g., into the upper interior portion of reaction vessel 4), downward flow of inert gas from the upper interior portion of reaction vessel 4 (e.g., through the headspace), and purging (e.g., flushing, displacement, venting, etc.) of volatized deprotecting agent and inert gas from the upper interior portion (e.g., from the headspace) of reaction vessel 4, is schematically depicted by the arrows in FIG. 1A.

In certain embodiments, as depicted in FIG. 1B, the process can include providing (e.g., directing) pressurized inert gas from an inert gas source (such as inert gas source designated as 100 in FIG. 2B) through flow path 218, opening 208, the inner interior space of spray head 220, and out openings 222 into outer interior space 7 of reaction vessel 4 (e.g., into the headspace above reactants 30). FIG. 1B also depicts embodiments wherein the spray head 220 directs (e.g., sprays) inert gas through openings 222 at an angle (e.g., spray pattern) schematically depicted by dashed lines 224 towards side wall 6 of the reaction vessel 4. This can facilitate a washing effect, wherein the inert gas can contact the side wall and “wash” condensed deprotecting agent toward reactants in the lower portion of the interior of the reaction vessel 4.

As the pressurized inert gas flows through the upper interior portion (e.g., through the headspace above the reactants) of reaction vessel 4 including volatized (evaporated) deprotecting agent, the inert gas purges (e.g., flushes, displaces, replaces, vents, discharges, etc.) volatized deprotecting agent out of reaction vessel 4 through opening 206 into flow path 216. Again, the pressurized inert gas in effect displaces the volatized deprotecting gas from the headspace of the reaction vessel, which may reduce residence time and minimize condensation of the deprotecting agent on the walls of the reaction vessel.

Gas flow (movement) in reaction vessel 4, including upward flow of volatized deprotecting agent (e.g., pyrrolidine) from reactants 30 in the lower interior portion of reaction vessel 4 into the headspace above the reactants (e.g., into the upper interior portion of reaction vessel 4), flow of inert gas from spray head 220 through openings of the plurality of openings 222 (e.g., through openings of the plurality of openings 222 into and through the headspace and angled flow towards side wall 6), and purging (e.g., flushing, displacing, venting, etc.) of volatized deprotecting agent and inert gas from the upper interior portion of reaction vessel 4 (e.g., from the headspace), is schematically depicted by the arrows and dashed lines in FIG. 1B.

In certain embodiments, the process may include introducing (directing) an inert gas into the lower portion (e.g., into the lower interior portion) of reaction vessel 4, in addition to or as an alternative to introducing (directing) an inert gas into the upper interior portion (e.g., into the headspace) of the reaction vessel through an opening in an upper portion of the reaction vessel, such as described herein. For example, referring to FIGS. 1A and 1B, the process can include directing a pressurized inert gas from an inert gas source (which can be the same as or different from an inert gas source of an inert gas introduced into the upper interior portion of the reaction vessels when present) through flow path 26 and introducing the pressurized inert gas into the lower interior portion of reaction vessel 4 through opening 16. The inert gas may flow upwardly from the lower interior portion of the reaction vessel (e.g., upwardly through reactants 30) into/through the upper interior portion (e.g., the headspace) of the reaction vessel 4 including volatized (evaporated) deprotecting agent. As the inert gas flows upwardly, the inert gas may purge (e.g., flush, displace, replace, vent, discharge, etc.) volatized deprotecting agent from the upper interior portion (e.g., the headspace) of the reaction vessel 4 through opening 14 into flow path 24. Again, in this manner, the inert gas may displace the volatized deprotecting gas from the headspace of the reaction vessel, which may reduce residence time and minimize condensation of the deprotecting agent on the walls of the reaction vessel.

The inert gas flow in the lower portion of the reaction vessel can also agitate (mix, bubble, etc.) the protected amino acid and the deprotecting agent reactants 30.

In some embodiments, the process may include introducing (directing) both a first pressurized inert gas into an upper interior portion (e.g., the headspace) of the reaction vessel including evaporated deprotecting agent and a second pressurized inert gas into a lower interior portion of a reaction vessel. As non-limiting examples, referring to FIGS. 1A and 1B, the process may include directing the first pressurized inert gas into the upper interior portion of reaction vessel 4 through a first opening located in an upper portion of the reaction vessel, such as opening 10 of FIG. 1A or opening 208 and openings 222 of FIG. 1B, and directing the second pressurized inert gas into the lower interior portion of the reaction vessel 4 through a second opening located in a lower portion of the reaction vessel such as opening 16 of FIGS. 1A and 1B. The first pressurized inert gas may move through the upper interior portion (e.g., through the headspace) of the reaction vessel including evaporated deprotecting agent, and the second pressurized inert gas may move generally upwardly from the lower interior portion of the reaction vessel through reactants 30 and into/through the upper interior portion (e.g., the headspace) of the reaction vessel including evaporated deprotecting agent. The first pressurized inert gas and optionally the second pressurized inert gas may purge (e.g., flush, displace, replace, vent, discharge, etc.) volatized deprotecting agent from the upper interior portion (e.g., from the headspace) of the reaction vessel, for example, through a third opening located in an upper portion of the reaction vessel, such as opening 14 or 206 of FIGS. 1A and 1B, respectively.

The first inert gas (also referred to herein as the overhead inert gas) directed into and/or moving through the upper interior portion (e.g., the headspace above reactants 30) of reaction vessel 4 (e.g., the first inert gas directed through a first opening located in an upper portion of the reaction vessel, such as opening 10 of FIG. 1A or opening 208 and openings 222 of FIG. 1B) may have a higher pressure than the second inert gas introduced into the lower portion of the interior of reaction vessel 4. As a non-limiting example, the first (overhead) inert gas directed into and/or moving through the upper interior portion (e.g., moving through the headspace) of reaction vessel 4 can have a pressure from about 1 psi to about 25 psi. In some embodiments, the first (overhead) inert gas directed into and/or moving through the upper interior portion of reaction vessel 4 can have a pressure of about 1, 2, 3, 4, 5, 6, 7, 8, 9, 10, 11, 12, 13, 14, 15, 16, 17, 18, 19, 20, 21, 22, 23, 24, or 25 psi. Further, according to some embodiments, the first (overhead) inert gas directed into and/or moving through the upper interior portion of the reaction vessel 4 can have a pressure in a range from about any of the foregoing pressure values to about any other of the foregoing pressure values.

As a non-limiting example, the second inert gas introduced into the lower interior portion of the reaction vessel 4 can have a pressure that is less than the pressure of the first (overhead) inert gas directed into and/or moving through the upper interior portion of the reaction vessel 4. For example, the second inert gas may have a pressure from about 1 psi to about 25 psi, so long as the pressure of the second inert gas is less than the pressure of the first inert gas. In embodiments wherein an inert gas is introduced only into the lower interior portion of the reaction vessel (there is no inert gas introduced into an upper interior portion of the reaction vessel), the inert gas may also have a pressure from about 1 psi to about 25 psi.

In a non-limiting example, the first (overhead) inert gas directed into and/or moving through the upper interior portion (e.g., head space) of reaction vessel 4 can have a pressure of about 15 psi and the second inert gas introduced into the lower portion of the reaction vessel 4 can have a pressure that is less than the pressure of the first (overhead) inert gas directed into and/or moving through the upper portion of the reaction vessel 4, such as a pressure of about 5 psi.

The process accordingly allows the use of deprotecting agents with relatively lower boiling points at higher temperatures to accelerate reaction times, while minimizing (reducing) adverse effects associated with using a low boiling point, readily volatized reactant. For example, the process can improve peptide purity, even for longer peptide chains. In addition, the process can improve peptide purity even using pyrrolidine as the deprotecting agent, despite significant evaporating observed due to its lower boiling point. This can also provide benefits of faster deprotection reactions.

After deprotecting is completed, the inert gas flow is stopped. The deprotecting agent is then drained, for example, through opening 16.

The present disclosure also relates to solid phase peptide synthesis processes including one or more deprotecting steps using an inert gas to purge volatized deprotecting agent from the reaction vessel (e.g., to flush volatized deprotecting agent from the headspace of the interior of the reaction vessel) in accordance with any of the embodiments of the deprotection step as described in more detail herein (e.g., SPPS processes including one or more deprotecting steps in accordance with the first embodiment and/or one or more deprotecting steps in accordance the second embodiment). The solid phase peptide synthesis process of the present disclosure further includes coupling amino acid(s), and/or washing, for example washing after deprotecting and/or coupling steps. Solid phase peptide synthesis coupling and washing steps and systems for conducting the same are generally known in the art and accordingly are not described in detail herein.

As noted herein, conventional SPPS processes require multiple washing steps between deprotection and coupling steps (e.g., after deprotection and before coupling) to remove residual deprotecting agent. In some embodiments of the present disclosure (e.g., SPPS processes including a deprotection step according to the first embodiment as described herein), a washing liquid (e.g., a solvent such as but not limited to dimethylformamide (DMF), methanol and/or isopropanol) can be added to the vessel for a washing step after deprotection. The washing step may be carried out repetitively (e.g., with five repetitions). The present disclosure is not limited to five washing steps, and fewer (e.g., one, two, three, or four) or more than five washing steps can be used.

Washing steps, however, can require the use of large amounts of solvent, necessitate solvent recovery and disposal, etc. This can increase material costs and peptide synthesis times, decrease efficiencies, etc. In addition, multiple washing steps may be less effective in preventing unwanted reactions and reducing impurities as peptide length increases, which can make it difficult to synthesize longer peptides with purities acceptable for downstream applications.

In some embodiments (e.g., SPPS processes including a deprotection step according to the second embodiment as described herein), the present disclosure is directed to SPPS processes including a deprotecting step followed by a coupling step, wherein the SPPS process does not include a washing step after the deprotecting step and before the associated coupling step. Stated differently, SPPS processes of the present disclosure including a deprotection step(s) according to the second embodiment may eliminate one or more washing steps (e.g., may eliminate all washing steps) between a deprotection step and its associated coupling step (i.e., the coupling step immediately following the deprotection step). This can provide benefits such as improved process efficiencies, energy savings, reduced amounts of solvent required in the SPPS process, reduced material costs, reduced solvent disposal issues, etc.

For example, SPPS processes of the present disclosure including a deprotection step(s) according to the second embodiment may include a series of deprotection-coupling cycles, wherein one or more (e.g., all) washing step(s) are eliminated (e.g., there is no washing step) between the deprotection step and the coupling step of at least one of the deprotection-coupling cycles of the SPPS process. In other examples, the SPPS processes of the present disclosure including a deprotection step(s) according to the second embodiment may include a series of deprotection-coupling cycles, wherein one or more (e.g., all) washing step(s) are eliminated (e.g., there is no washing step) between the deprotection step and the coupling step of more than one of the deprotection-coupling cycles, for example for half of the deprotection-coupling cycles, for example for a majority of the deprotection-coupling cycles, and as another example for all of deprotection-coupling cycles, of the SPPS process.

In yet other embodiments, the present disclosure is directed to SPPS processes including a deprotecting step(s) according to the second embodiment followed by a coupling step, wherein the SPPS process includes one or more washing steps (e.g., one, two, three, four, five, etc. washing steps) using a washing composition (e.g., a solvent) after the deprotecting step and before the associated coupling step. In contrast to conventional washing steps, however, the washing step(s) of this embodiment uses reduced amounts of solvent. More specifically, the washing step(s) after deprotection and before coupling may include washing the interior of the reaction vessel one or more times (e.g., one, two, three, four, five, etc. times) using a washing composition (e.g., a solvent) in an amount that is less than the total volume of the deprotecting composition used in the deprotection step. For example, the washing step can include washing the interior of the reaction vessel one or more times (e.g., one, two, three, four, five, etc. times) using a washing composition (e.g., a solvent) in an amount that is less than ½ of the total volume of the deprotecting composition used in the deprotection step. As another non-limiting example, the process can include washing the interior of the reaction vessel one or more times (e.g., one, two, three, four, five, etc. times) using a washing composition (e.g., a solvent) in an amount that is less than ⅓ of the total volume of the deprotecting composition used in the deprotection step. In addition, the SPPS process may include a series of deprotection-coupling cycles, wherein one or more of (e.g., half of, a majority of, or all of) the deprotection-coupling cycles include one or more washing steps (e.g., one, two, three, four, five, etc. washing steps) between the deprotection step and the coupling step, and wherein the washing step(s) uses a washing composition (e.g., solvent) in an amount that is less than the total volume of the deprotecting composition used in the deprotection step (for example, in an amount that is less than ½ of the total volume of the deprotecting composition, and as another example in an amount that is less than ⅓ of the total volume of the deprotecting composition). The volume of solvent used for each washing step and/or the total volume of solvent used for all washing steps of a given deprotection-coupling cycle (after deprotection and before the next coupling step) may be less than the total volume of the deprotecting composition used in the deprotection step (for example, an amount that is less than ½ of the total volume of the deprotecting composition, and as another example an amount that is less than ⅓ of the total volume of the deprotecting composition).

When used, the washing composition (washing liquid) can include a solvent such as but not limited to dimethylformamide (DMF), methanol and/or isopropanol.

When a washing step is used, in some embodiments, the washing liquid (e.g., solvent) can be introduced into the reaction vessel via a suitable opening into an upper interior portion of the reaction vessel, such as opening 10 of FIG. 1A, and/or using a different spray head or the same spray head (e.g., spray head 220 of FIG. 1B) used to introduce the inert gas into the reaction vessel during the deprotecting step described herein. As a non-limiting example, as depicted in FIG. 1B, the process can include providing (e.g., directing) solvent from a solvent source (not illustrated in FIG. 1B) through flow path 218, opening 208, the inner interior space of spray head 220, and out openings 222 into the outer interior space 7 of reaction vessel 4. As also schematically depicted in FIG. 1B, in some embodiments, the spray head 220 can direct (e.g., spray) solvent through openings 222 at an angle (e.g., spray pattern) schematically depicted by dashed lines 224 towards side wall 6 of the reaction vessel 4, which can facilitate washing deprotecting agent condensed on the side wall downwardly toward the lower interior portion of reaction vessel 4.

When a washing step is included, the washing solution can then be removed in a second draining step, after which a coupling step can be initiated in accordance with known processes.

The solid phase peptide synthesis process of the present disclosure more specifically can include: deprotecting a first amino acid (e.g., removing a protecting group of a first protected amino acid), which first amino acid can be linked directedly or indirectly to a solid phase resin, to form a deprotected amino acid; optionally washing the deprotected amino acid; coupling a second amino acid to the deprotected amino acid to form a peptide from the first and second amino acids; and repeating the deprotecting, washing, and/or coupling steps to form a peptide comprising the first, second, and successive plurality of amino acids, wherein one or more of the deprotecting steps employ the inert gas purging (flushing) step described herein (e.g. in accordance with the first and/or second embodiments of the deprotection steps described herein).

In some embodiments, the solid phase peptide synthesis process can include: deprotecting a first protected amino acid (e.g., removing a protecting group of a first protected amino acid) to form a deprotected amino acid; washing the deprotected amino acid; coupling a second amino acid to the deprotected amino acid to form a peptide from the first and second amino acids; and repeating the deprotecting, washing, and coupling steps to form a peptide comprising the first, second, and successive plurality of amino acids,
wherein the deprotecting and coupling steps take place in a reaction vessel (e.g., in the same reaction vessel such as reaction vessel 4 as described in more detail herein) and wherein one or more of the deprotecting steps include:
heating a protected amino acid and a deprotecting agent in a lower interior portion of the reaction vessel during the deprotection step, wherein the heating step volatizes deprotecting agent into an upper interior portion (e.g., the headspace) of the reaction vessel; and
directing a first inert gas into the upper interior portion of the reaction vessel through a first opening in an upper portion of the reaction vessel, through the upper interior portion (e.g., through the headspace) of the reaction vessel including volatized deprotecting agent and out of the upper interior portion of the reaction vessel through a second opening in the upper portion of the reaction vessel during the heating step to purge (flush) volatized deprotecting agent from the interior of the reaction vessel.

In some embodiments, the solid phase peptide synthesis process can include: deprotecting a first protected amino acid (e.g., removing a protecting group of a first protected amino acid) to form a deprotected amino acid; coupling a second amino acid to the deprotected amino acid to form a peptide from the first and second amino acids; and repeating the deprotecting and coupling steps to form a peptide comprising the first, second, and successive plurality of amino acids,
wherein the deprotecting and coupling steps take place in a reaction vessel (e.g., in the same reaction vessel such as reaction vessel 4 as described in more detail herein),
wherein one or more of the deprotecting steps employ a deprotecting composition including a deprotecting agent in an amount of about 5 vol % or less, based on the total volume of a deprotecting composition, as described in more detail herein (e.g., in accordance with the deprotecting process of the second embodiment) and/or
wherein one or more of the deprotecting steps employ an inert gas purging (e.g., headspace flushing) step to remove evaporated deprotecting agent from an upper interior portion (e. g., from the headspace) of the reaction vessel, also as described in more detail herein (e.g., in accordance with the deprotecting process of the second embodiment).

For example, the SPPS process including a deprotecting step according to the second embodiment may include directing an inert gas through the reaction vessel (e.g., through the upper interior portion of the reaction vessel (through the headspace) including evaporated deprotecting agent) to remove evaporated deprotecting agent from the interior of the reaction vessel (e.g., from the upper interior portion of the reaction vessel, from the headspace).

More specifically, the SPPS process including a deprotecting step according to the second embodiment may include directing (introducing, supplying, etc.) inert gas into the upper interior portion of the reaction vessel through a first opening located in an upper portion of the reaction vessel and through the upper interior portion (e.g., the headspace) of the reaction vessel including evaporated deprotecting agent); and venting (flushing) the inert gas and evaporated deprotecting agent from the upper interior portion (e.g., the headspace) of the reaction vessel through a second opening also located in an upper portion of the reaction vessel.

As another example, the SPPS process including a deprotecting step according to the second embodiment may include directing (introducing, supplying, etc.) inert gas into the lower interior portion of the reaction vessel through an opening located in a lower portion of the reaction vessel and generally upwardly from the lower interior portion of the reaction vessel (e.g., generally upwardly through reactants in the lower interior portion of the reaction vessel) into/through the upper interior portion (e.g., the headspace) of the reaction vessel including evaporated deprotecting agent; and venting (flushing) the inert gas and evaporated deprotecting agent from the upper interior portion (e.g., the headspace) of the reaction vessel through another opening located in an upper portion of the reaction vessel.

As yet another example, the SPPS process including a deprotecting step according to the second embodiment may include directing (introducing, supplying, etc.) inert gas into both the upper interior portion of the reaction vessel and into the lower interior portion of the reaction vessel; and venting (flushing) the inert gas and evaporated deprotecting agent from the upper interior portion (e.g., the headspace) of the reaction vessel. More specifically, the deprotecting step according to the second embodiment may include directing (introducing, supplying, etc.) inert gas into the upper interior portion of the reaction vessel through a first opening located in an upper portion of the reaction vessel and through the upper interior portion (e.g., the headspace) of the reaction vessel including evaporated deprotecting agent and also directing inert gas into the lower interior portion of the reaction vessel through a second opening located in a lower portion of the reaction vessel and optionally upwardly from the lower interior portion of the reaction vessel (e.g. optionally generally upwardly through reactants in the lower interior portion of the reaction vessel and into/through the upper interior portion (e.g., the headspace) of the reaction vessel including evaporated deprotecting agent); and venting the inert gas and the evaporated deprotecting agent from the upper interior portion of the reaction vessel through a third opening located in an upper portion of the reaction vessel.

In the SPPS processes including a deprotecting step according to the second embodiment, the solid phase peptide synthesis process may not include a washing step between sequential deprotecting and coupling steps. In other SPPS processes including a deprotecting step according to the second embodiment, the solid phase peptide synthesis process may include one or more washing steps after a deprotecting step and before a sequential coupling step, the washing step using reduced amounts of washing liquid (e.g., solvent) as described in more detail herein (e.g., using solvent in an amount that is less than the total volume of the deprotecting composition used in the deprotection step, for example, in an amount that is less than ½ of the total volume of the deprotecting composition, and as another example in an amount that is less than ⅓ of the total volume of the deprotecting composition). For example, the solid phase peptide synthesis process may omit one or more (e.g., all) washing steps between one or more of the sequential deprotecting-coupling steps. As another example, the solid phase peptide synthesis process may include washing a deprotected amino acid using reduced amounts of washing liquid (e.g., solvent) as described in more detail herein (e.g., using solvent in an amount that is less than the total volume of the deprotecting composition used in the deprotection step, for example, in an amount that is less than ½ of the total volume of the deprotecting composition, and as another example in an amount that is less than ⅓ of the total volume of the deprotecting composition) after deprotection and before an associated coupling step for one or more of the sequential deprotecting-coupling steps.

The solid phase peptide synthesis process can further include, prior to coupling, activating chemical group(s) on the second amino acid (and successive amino acid(s)) using processes and agents known in the art to prepare the second (and successive) amino acid(s) for coupling with the first (and sequential) amino acid(s).

An amino acid activating agent may be used to activate the amino acid (e.g., convert the acid group of the amino acid into an activated form) prior to a coupling step. Any suitable amino acid activating agent may be used. Examples of an amino acid activating agent include without limitation carbodiimides and/or onium salt activating agents. The amino acid activating agent comprises, in some embodiments, a carbodiimide, such as but not limited to N,N′-diisopropylcarbodiimide (DIC), N,N′-dicyclohexylcarbodiimide (DCC), 1-ethyl-3-(3-dimethylaminopropyl) carbodiimide (EDC), and the like and combinations thereof. In certain embodiments, the amino acid activating agent comprises an onium activating agent, such as but not limited to benzotriazol-1-yloxytripyrrolidinophosphonium hexafluorophosphate (PyBOP), O-(benzotriazol-1-yl)-N,N,N′,N′-tetramethyluronium hexafluorophosphate (HBTU), 2-(7-aza-1H-benzotriazole-1-yl)-1,1,3,3-tetramethyluronium hexafluorophosphate (HATU), 1-[(1-(cyano-2-ethoxy-2-oxoethylideneaminooxy) dimethylaminomorpholino)] uronium hexafluorophosphate (COMU), and the like and combinations thereof.

Still further, in exemplary embodiments, the solid phase peptide synthesis process can include applying microwave energy during one or more of the solid phase peptide synthesis steps, for example, during the deprotecting and/or coupling steps.

In exemplary embodiments, the solid phase peptide synthesis process can further include cleaving the peptide from the solid phase resin after the deprotecting, washing, and/or coupling steps.

The skilled artisan will understand how to join or couple amino acids to form a chain. Processes and agents for cleaving a peptide from a solid phase resin are also well known in the art. Accordingly, a detailed discussion of processes known in the art for joining amino acids to form a peptide and/or cleaving a peptide from a solid phase resin is not provided.

The present disclosure also relates to a system for solid phase peptide synthesis. FIG. 2A is a schematic flow diagram depicting selected portions of an exemplary solid phase peptide synthesis system in accordance with embodiments of the present disclosure. FIG. 2B is a schematic flow diagram depicting selected portions of another exemplary peptide synthesis system in accordance with other embodiments of the present disclosure

Generally, the elements illustrated in both FIG. 1A and FIG. 2A will carry the same reference numerals. Similarly, generally the elements illustrated in both FIG. 1B and FIG. 2B will carry the same reference numerals. Also, except where indicated otherwise, elements illustrated in both FIG. 2A and FIG. 2B will carry the same reference numerals.

The peptide synthesis system of FIGS. 2A and 2B is designated generally as 2. Peptide synthesis system 2 includes reaction vessel 4 as discussed herein. Peptide synthesis system 2 also includes a plurality of reagent containers located in a position upstream of reaction vessel 4 in fluid communication with reaction vessel 4.

For example, as depicted in FIGS. 2A and 2B, system 2 can include a plurality of solid support containers 50a, 50b, and 50c in fluid communication with a flow path 52 fluidly connecting the solid support containers and reaction vessel 4 for delivering a solid support (e.g., a solid resin having a protected amino acid linked thereto) from the solid support container(s) to reaction vessel 4. Flow paths 51a, 51b, and 51c fluidly connect solid support containers 50a, 50b, and 50c, respectively, and flow path 52.

In certain embodiments, flow path 52 can be in direct fluid communication with reaction vessel 4, e.g., via opening 12 or 204 of FIG. 1A or 1B, respectively. In certain embodiments, as depicted schematically in FIG. 2A, the system can include a rotary valve 140 that can rotate among multiple positions (e.g., two positions) to fluidly connect reaction vessel 4 through opening 12, flow path 22, and flow path 52 with solid support containers 50a, 50b, and 50c. In certain other embodiments, as depicted schematically in FIG. 2B, rotary valve 140 can rotate among multiple positions (e.g., two positions, or more) to fluidly connect reaction vessel 4 through opening 204, flow path 214, and flow path 52 with solid support containers 50a, 50b, and 50c.

As another example, in certain embodiments, as depicted schematically in FIGS. 2A and 2B, system 2 can include a plurality of amino acid containers 60a, 60b, and 60c in fluid communication with flow path 62 (FIG. 2A) or flow path 212 (FIG. 2B) fluidly connecting the amino acid containers and reaction vessel 4 for delivering protected amino acid from the amino acid container(s) to reaction vessel 4. Flow paths 61a, 61b, and 61c fluidly connect amino acid containers 60a, 60b, and 60c, respectively, with flow path 62 (FIG. 2A) or flow path 212 (FIG. 2B). In some embodiments, flow path 62 and/or flow path 212 can be in direct fluid communication with reaction vessel 4, e.g., via opening 10 or 202 of FIG. 1A or 1B, respectively. In some embodiments, the system can include one or more additional valves and/or flow paths, as schematically depicted in FIGS. 2A and 2B and as described in more detail below.

As another example, as schematically depicted in FIGS. 2A and 2B, system 2 can include a deprotecting agent container 70 in fluid communication with a flow path 72 fluidly connecting the deprotecting agent container and reaction vessel 4 for delivering deprotecting agent from the deprotecting container to reaction vessel 4. In some embodiments, flow path 72 can be in direct fluid communication with reaction vessel 4, e.g., via opening 16 of FIGS. 1A and 1B. In some embodiments, the system can include one or more additional valves and/or flow paths, as schematically depicted in FIGS. 2A and 2B and as described in more detail below.

As yet another example, as schematically depicted in FIGS. 2A and 2B, system 2 can include a solvent container 80 in fluid communication with a flow path 82 fluidly connecting the solvent container and reaction vessel 4 for delivering solvent from the solvent container to reaction vessel 4. In some embodiments, flow path 82 can be in direct fluid communication with reaction vessel 4, e.g., via opening 10 or 208 of FIG. 1A or 1B, respectively. In some embodiments, the system can include one or more additional valves and/or flow paths, as schematically depicted in FIGS. 2A and 2B and as described in more detail below. FIG. 2B also schematically depicts embodiments wherein solvent can be introduced into reaction vessel 4 using at least a portion of the same flow path used to introduce an inert gas, e.g., via flow path 218, opening 208, spray head 220 and plurality of openings 222, as described in more detail herein.

As yet another example, as schematically depicted in FIGS. 2A and 2B, system 2 can include an additional reagent container 90, which can be for example an activating agent container, in fluid communication with a flow path 92 (FIG. 2A) or flow path 210 (FIG. 2B) fluidly connecting the additional reagent container and reaction vessel 4 for delivering an additional reagent such as an activating agent from the additional reagent container to reaction vessel 4. In some embodiments, flow path 92 or 210 can be in direct fluid communication with reaction vessel 4, e.g., via opening 10 or 200 of FIGS. 1A and 1B, respectively. In some embodiments, the system can include one or more additional valves and/or flow paths, as schematically depicted in FIGS. 2A and 2B and as described in more detail below.

The skilled artisan will appreciate that the number of reaction vessels, solid support containers, amino acid containers, deprotecting agent containers, solvent containers, and/or other reagent containers and associated flow paths, as well as the manner in which these elements are connected, can vary and is not limited to the depiction thereof in FIG. 2A and FIG. 2B. The skilled artisan will also understand that the peptide synthesis system can include various subsystems associated with the aforementioned containers, flow paths, and/or reaction vessel(s), including for example flow paths, valves, filters, gauges, monitors, controllers, etc., to direct the flow of materials into and/or out of containers and/or the reaction vessel(s) at appropriate stages of the solid phase peptide synthesis process. Such subsystems are well known in the art and will not be described in detail herein.

System 2 is also associated with a heating source (not shown) such as a microwave source, and associated elements such as microwave guides and/or microwave cavities, for heating reaction vessel 4, as described herein. Heating sources, including microwave heating sources, and associated elements such as microwave guides and/or microwave cavities, and the use thereof in solid phase peptide synthesis processes and systems, are also well known in the art and are not described in more detail herein.

System 2 is schematically depicted as operating in an amino acid deprotecting step of a solid phase peptide synthesis, such as described herein with reference to FIG. 1A and FIG. 1B. In this operational state, reactants including a protected amino acid and a deprotecting agent have already been delivered to reaction vessel 4 (are already present in reaction vessel 4).

The protected amino acid is attached to a solid support. The protected amino acid can be directly attached to the solid support, such as delivered to reaction vessel 4 via flow path 52 from one or more of solid support containers 50a, 50b, and 50c. Alternatively, the protected amino acid can be indirectly attached to a solid support (e.g., can be attached to another amino acid or to a peptide chain, wherein the other amino acid or peptide is attached to the solid support).

System 2 further includes an inert gas source 100 located in a position upstream of reaction vessel 4 in fluid communication with reaction vessel 4. In certain embodiments, as depicted schematically in FIGS. 2A and 2B, a flow path 102 fluidly connects inert gas source 100 and reaction vessel 4 and delivers inert gas supplied from inert gas source 100 into the upper interior portion of reaction vessel 4 via opening 10 (FIG. 1A) or opening 208 (FIG. 1B) described herein. In certain embodiments, system 2 includes a valve 104 in fluid communication with flow path 102 located between inert gas source 100 and opening 10 (FIG. 1A) or opening 208 (FIG. 1B), wherein valve 104 has an open position and a closed position relative to flow path 102.

Valve 104 is shown in an open position in FIG. 2A and FIG. 2B relative to flow path 102. In the open position, valve 104 allows flow of pressurized inert gas from inert gas source 100 through flow path 102, flow path 20 (FIG. 1A) or flow path 218 (FIG. 1B), and opening 10 (FIG. 1A) or opening 208 and spray head 220 (FIG. 1B), and into the interior upper portion of reaction vessel 4 (e.g., into the reaction vessel headspace). In this manner, the system can continuously and/or intermittently direct an overhead source of pressurized inert gas into the upper portion of the interior of the reaction vessel during the heating and/or deprotecting step to purge (flush, vent) volatized deprotecting agent out of the reactor headspace, in accordance with the process described herein.

In contrast, when valve 104 is in a closed position relative to flow path 102, valve 104 can prevent the flow of pressurized inert gas from the inert gas source through flow path 102, flow path 20 (FIG. 1A) or flow path 218 (FIG. 1B), and opening 10 (FIG. 1A) or opening 208 and spray head 220 (FIG. 1B).

The system can also include a flow path 106 fluidly connecting inert gas source 100 and opening 16 in the lower portion of reaction vessel 4. The system can also include a valve 108 in fluid communication with flow path 106 located between inert gas source 100 and opening 16, wherein valve 108 has an open position and a closed position relative to flow path 106.

When in the open position relative to flow path 106, valve 108 allows flow of pressurized gas from inert gas source 100 through flow path 106, flow path 26, and opening 16 into the interior lower portion of reaction vessel 4. As described herein, in the deprotecting process, in this manner pressurized inert gas can be directed into the lower portion of the reaction vessel to agitate (e.g., stir, bubble) the reactants and/or flush evaporated deprotecting agent from the headspace of the reaction vessel. The closed position of second valve 108 relative to flow path 106 prevents flow of pressurized inert gas from inert gas source 100 through flow path 106, flow path 26, and opening 16 into the interior lower portion of reaction vessel 4.

In certain embodiments, flow paths 102 and 106 can be fluidly connected via a pressure regulator, designated in FIG. 2A and FIG. 2B as 110, to a single inert gas source 100. Alternatively, flow paths 102 and 106 can fluidly connect the reaction vessel to at least two different inert gas sources.

When present, pressure regulator 110 can be located in a downstream position from inert gas source 100 and an upstream position from valves 104 and 108. In these embodiments, pressure source 100 directs inert gas to pressure regulator 110, which supplies pressurized inert gas to flow path 102 having a higher pressure (the “high pressure” inert gas) than inert gas supplied to flow path 106 (the “low pressure” inert gas). For example, without being limited thereto, pressure regulator 110 can supply “high pressure” inert gas having a pressure of about 1 psi to about 25 psi to flow path 102. Pressure regulator 110 can also supply “low pressure” inert gas having a pressure that is less than the pressure of the “high pressure” inert gas supplied to flow path 102.

Pressure regulators are also well known in the art and the skilled artisan will understand how to use the same in system 2 to provide a high pressure inert gas and a low pressure inert gas as discussed herein.

As also schematically depicted in FIGS. 2A and 2B (and FIGS. 1A and 1B), in exemplary embodiments, the system can include flow path 24 (FIGS. 1A and 2A) or flow path 216 (FIGS. 1B and 2B) located in a downstream position from opening 14 (FIG. 1A) or opening 206 (FIG. 1B) of reaction vessel 4. Flow paths 24 and 216 may function as a gaseous waste flow path to allow the purging (venting, flushing) of gaseous waste (e.g., volatized deprotecting agent, inert gas, etc.) during the deprotecting process described herein from the upper interior portion (headspace) of reaction vessel 4.

In certain embodiments, the system includes a valve 120 in fluid communication with flow path 24 or flow path 216. Valve 120 has an open position and a closed position relative to flow path 24 or flow path 216. The open position of valve 120 allows flow of gas from the upper interior portion (headspace) of reaction vessel 4 through opening 14 or opening 206 and flow path 24 or flow path 216 to a waste recovery zone, such as a vent and/or waste container (not shown) to permit purging/venting of gas from reaction vessel 4. The closed position of valve 120 prevents flow of gas out of the upper interior portion (headspace) of the reaction vessel through opening 14 or opening 206.

FIGS. 2A and 2B illustrate certain embodiments of the system in an operational state wherein both valve 104 and valve 120 are in an open position with respect to flow path 102, flow path 20 or 218, and flow path 24 or flow path 216, respectively. This corresponds to the positions that could be used during the (heated) deprotecting process described herein. The simultaneously open positions of valve 104 and valve 120 relative to flow path 102, flow path 20 or 218, and flow path 24 or flow path 216, respectively, allow an overhead supply of pressurized inert gas to flow continuously and/or intermittently through reaction vessel 4 (for example, flow into vessel 4 through opening 10 in the vessel headspace and out of vessel 4 through a separate opening 14 or as another example flow into vessel 4 through opening 208, spray head 220 and openings 222 toward side wall 6 and into the vessel headspace and out of vessel 4 through a separate opening 206) to purge (flush) volatized reactants present in the headspace during the heated deprotecting step.

In certain embodiments, system 2 can include a valve 122 in series with valve 104 and a flow path 124 positioned (disposed) between and fluidly connecting valve 104 and valve 122. Valve 122 is in fluid communication with flow path 102 and has an open position and a closed position relative to flow path 102. When valve 122 is present and valve 122 and valve 104 are in an open position relative to flow path 102, such as depicted in FIG. 2A and FIG. 2B, inert gas source 100, pressure regulator 110, flow path 102, valve 104, flow path 124, valve 122, flow path 20 (FIG. 2A) or flow path 218 (FIG. 2B), opening 10 (FIG. 1A/2A) or opening 208 and spray head 220 (FIG. 2A/2B), and reaction vessel 4 can be fluidly connected (in fluid communication).

In certain embodiments, valve 104 can be a rotary valve which can rotate between multiple positions to fluidly connect one flow path selected from a plurality of flow paths with reaction vessel 4. As a non-limiting example, FIG. 2A depicts rotary valve 104 capable of rotating between four positions to fluidly communicate with flow path 102, 62, 82, or 92, depending on the open or closed position of the valve. For example, FIG. 2A schematically depicts rotary valve 104 in an open position with respect to flow path 102 but in a closed position with respect to flow paths 62, 82, and 92. As another non-limiting example, FIG. 2B depicts rotary valve 104 capable of rotating between two positions to fluidly communicate with flow path 102 or 82, depending on the open or closed position of the valve. For example, FIG. 2B schematically depicts rotary valve 104 in an open position with respect to flow path 102 but in a closed position with respect to flow path 82. The open/closed positions of rotary valve 104 relative to the different flow paths can be selected depending on the stage of the peptide synthesis process and the reactants, etc. to be delivered to reaction vessel 4. Rotary valve 104 (and other valves discussed herein) can be operated as known in the art.

Other valves of the system can also be rotary valves. For example, as noted herein, in certain embodiments, system 2 can include a flow path 124 positioned (disposed) between and fluidly connecting valve 104 and valve 122. In this embodiment, as depicted schematically in FIG. 2A and FIG. 2B, valve 122 in series with valve 104 can rotate among multiple positions (e.g., valve 122 between two positions in FIGS. 2A and 2B and valve 104 between four positions as schematically depicted in FIG. 2A and between two positions as schematically depicted in FIG. 2B) to fluidly connect, for example, inert gas source 100 and reaction vessel 4 via flow path 102, flow path 124, flow path 20 or flow path 218, and opening 10 or opening 208. Alternatively, as shown in FIG. 2A, valve 122 in series with valve 104 can rotate among multiple positions (e.g., two positions and four positions, respectively) to fluidly connect, for example, one or more amino acid containers 60a, 60b, and 60c and reaction vessel 4 via flow path 62, flow path 124, flow path 20, and opening 10; solvent container 80 and reaction vessel 4 via flow path 82, flow path 124, flow path 20, and opening 10; or reagent container 90 and reaction vessel 4 via flow path 92, flow path 124, flow path 20, and opening 10. Also in some embodiments, as shown in FIG. 2B, valve 122 in series with valve 104 can rotate among multiple positions (e.g., two positions) to fluidly connect, for example, solvent container 80 and reaction vessel 4 via flow path 82, flow path 124, flow path 218, and opening 208 and spray head 222.

As another example, in certain embodiments, as depicted schematically in FIG. 2A and FIG. 2B, valve 108 can be a rotary valve rotating among multiple positions (e.g., three positions) to fluidly connect reaction vessel 4 through opening 16, flow path 26, and flow path 72, flow path 106 or a flow path 132 with deprotecting agent container 70, inert gas source 100, or a waste container 130, respectively, depending on the position of valve 108. For example, FIG. 2A and FIG. 2B schematically depict rotary valve 108 in an open position with respect to flow path 106 but in a closed position with respect to flow paths 72 and 132. This can be the position of rotary valve 108 during the heating and/or deprotecting process described herein, wherein low pressure inert gas is directed (bubbled) into a lower portion of reaction vessel 4 to stir the reactants and/or flush evaporated deprotecting agent from the headspace of the reaction vessel.

As another example, as discussed herein, in certain embodiments, as also depicted schematically in FIG. 2A, the system can include a rotary valve 140 that can rotate among multiple positions (e.g., two positions) to fluidly connect reaction vessel 4 through opening 12, flow path 22, and flow path 52 with solid support containers 50a, 50b, and 50c. Alternately, rotary valve 140 can rotate among multiple positions (e.g., two positions) to fluidly connect reaction vessel 4 through opening 12, flow path 22, and a flow path 152, wherein flow path 152 is in fluid communication with a plurality of flow paths 151a, 151b, and 151c, which in turn fluidly connect flow path 152 with a plurality of product containers 150a, 150b, and 150c, respectively. This can allow the passage of product (e.g., peptide and/or peptide linked to a solid support) from reaction vessel 4 into container(s) 150a, 150b, and 150c. Again, the skilled artisan will appreciate that the number of product containers 150a, 150b, and 150c, and corresponding flow paths 151a, 151b, and 151c, can vary and is not limited to the number depicted in FIG. 2A.

As another example, as discussed herein, in certain embodiments, as also depicted schematically in FIG. 2B, the system can include a rotary valve 140 that can rotate among multiple positions (e.g., two positions) to fluidly connect reaction vessel 4 through opening 204, flow path 214, and flow path 52 with solid support containers 50a, 50b, and 50c. Alternately, rotary valve 140 can rotate among multiple positions (e.g., two positions) to fluidly connect reaction vessel 4 through opening 204, flow path 214, and flow path 152, wherein again flow path 152 is in fluid communication with a plurality of flow paths 151a, 151b, and 151c, which in turn fluidly connect flow path 152 with a plurality of product containers 150a, 150b, and 150c, respectively. Again, this can allow the passage of product (e.g., peptide and/or peptide linked to a solid support) from reaction vessel 4 into container(s) 150a, 150b, and 150c. Also again, the skilled artisan will appreciate that the number of product containers 150a, 150b, and 150c, and corresponding flow paths 151a, 151b, and 151c, can vary and is not limited to the number depicted in FIG. 2B.

Peptide synthesis system 2 can also include one or more flow paths, vents, containers, valves, controllers, and the like, for example, for the removal of waste (e.g., excess reactants, solvents, etc.) from the peptide synthesis system. The waste can be in gas, liquid and/or solid form and the skilled artisan will appreciate appropriate types of flow paths and containers for removing the same from the peptide synthesis system. For example, in certain embodiments, as discussed herein, waste container 130 can be fluidly connected to reaction vessel 4 via flow path 132 and rotary valve 108 when in the appropriate open position to allow the passage of waste products from reaction vessel 4 to waste container 130. As another example, in certain embodiments, as discussed herein, the open position of valve 120 can allow flow of gas from the upper interior portion (headspace) of reaction vessel 4 through opening 14 or opening 206 and flow path 24 or flow path 216 to a waste recovery zone, such as a vent and/or waste container (not shown) to permit purging/venting of gas from reaction vessel 4.

Thus, generally, FIGS. 2A and 2B illustrate an exemplary system for the delivery of solvents, reactants (amino acids, solid phase resins, deprotecting agents, activators, etc.), gases, etc. from their respective sources to reaction vessel 4 and the further delivery of products and by-products (peptides, gas, liquid, and/or solid waste, etc.) from reaction vessel 4 to their respective destinations. It will be understood that the particular flow paths and valve locations illustrate, rather than limit, the present disclosure.

At least partially reiterating from above, the peptide synthesis system typically includes at least one controller operatively associated with, for example, numerous electrical components of the system (e.g., the microwave source, sensors, and solenoid and/or other motor-operated valves). The at least one controller can include one or more computers, computer data storage devices, programmable logic devices (PLDs) and/or application-specific integrated circuits (ASIC). A suitable computer can include one or more of each of a central processing unit or processor, computer hardware integrated circuits or memory, user interface, peripheral or equipment interface for interfacing with other electrical components of the system, and/or any other suitable features. The controller(s) can respectively communicate with electrical components of the system by way of suitable signal communication paths. In FIGS. 2A and 2B, representative signal communication paths associated with a controller are schematically depicted and designated by numerals *2 (signal communication paths) and *1 (controller), respectively. Processes of this disclosure can be controlled (e.g., at least partially controlled) in response to the execution of computer-based algorithms operatively associated with the at least one controller *1.

Solid phase peptide synthesis processes, including batch-based processes, are known and thus the present disclosure does not provide detailed information on the same. Reference is made, for example, to the pioneering work R. B. Merrifield (1963) “Solid Phase Peptide Synthesis I, The Synthesis of a Tetrapeptide,” J. Am. Chem. Soc. 85 (14), 2149-2154). Accordingly, a detailed discussion of solid phase peptide synthesis processes and systems is not provided.

Systems and processes suitable for conducting solid phase peptide synthesis, including batch-based processes, are also known. Exemplary systems for conducting solid phase peptide synthesis include, for example, the LIBERTY line of instruments commercially available from CEM Corporation of Matthews N.C. Exemplary US patents dealing with the subject of solid phase peptide synthesis include without limitation U.S. Pat. Nos. 7,393,920; 7,550,560; 7,563,865; 7,939,628; 7,902,488; 7,582,728; 8,153,761; 8,058,393; 8,426,560; 8,846,862; 9,211,522; 9,669,380; 10,052,607; 10,308,677; 10,125,163; 10,858,390; and 10,239,914. The contents of each of these are incorporated entirely herein by reference.

The deprotecting processes and/or SPPS processes of the present disclosure may be used in combination with SPPS processes (such as SPPS processes wherein the protected amino acids include Fmoc protecting groups) that do not include (that eliminate) washing after each coupling step and/or add deprotection base directly to a coupling solution without any draining after coupling, such as disclosed in, for example, U.S. Pat. Nos. 10,308,677; 10,125,163; 10,858,390; and 10,239,914. Such SPPS processes may be referred to generally as “High Efficiency SPPS (HE-SPPS).”

Next, the present invention will be described in more detail with reference to the following examples. It should be understood that these examples are provided for illustration only and are not to be in any way construed as limiting the present invention.

### Example 1

Synthesis of Thymosin with Headspace Cleaning During Deprotection

The peptide thymosin SDAAVDTSSEITTKDLKEKKEVVEEAEN-NH2 is synthesized (synthesis scale 0.05 mmol) using solid phase peptide synthesis with an automated microwave peptide synthesizer Liberty PRIME commercially available from CEM Corporation (Matthews, NC). Rink Amide ProTide LL (0.19 mmol/g substitution) is used as the solid phase resin support.

Deprotection is performed by adding 0.75 mL of a deprotection reagent including 40% pyrrolidine in dimethylformamide (DMF) to an undrained post-coupling mixture. Microwave power is regulated to provide a deprotection temperature of 110° C., and the deprotection steps are conducted for 50 seconds at 110° C. After each deprotection step, the amino acids are washed three times using 5 mL of DMF.

During deprotection, a pressurized nitrogen gas stream is continuously directed through the headspace of the reaction vessel at a pressure of about 15 psi (+/−3 psi) to continuously purge headspace gas from the reaction vessel in accordance with embodiments of the present disclosure described herein.

Coupling reactions are performed in the presence of a 10-fold molar excess of Fmoc-protected amino acids (AA) and activator/activator base diisopropylcarbodiimide (DIC) and ethyl (hydroxyimino) cyanoacetate (Oxyma), specifically AA/DIC/Oxyma (10:20:10) in 3.5 mL DMF. Coupling reactions are conducted under microwave conditions selected to provide a 30 second wait plus 90 seconds at 105° C.

Following completion of synthesis of the peptide, the peptide is cleaved from the solid phase using trifluoroacetic acid (TFA), triisopropyle silane (TIS), water (H₂O), and dioxa-1,8-octane-dithiol (DODT), specifically 5 mL of TFA/TIS/H₂0/DODT (92.5:2.5:2.5:2.5) for 30 minutes at 38° C. in a RAZOR cleavage system commercially available from CEM Corporation.

The peptide is analyzed using a Thermo Scientific Vanquish UPLC connected to an Extractive Plus Orbitrap Mass Spectrometer using acetonitrile/water with 0.1% TFA as the solvent system and C8 column (1.7 μm, 2.1×100 mm) or a Waters UPLC ACQUITY H-Class with a 3100 Single Quad Mass Spectrometer using acetonitrile/water with 0.1% TFA as the solvent system and C8 column (1.7 μm, 2.1×100 mm).

### Comparative Example 1

Synthesis of Thymosin without Headspace Cleaning During Deprotection

The peptide thymosin is synthesized under the same conditions described in Example 1, except that the deprotection steps did not include directing a pressurized nitrogen gas stream continuously through the headspace of the reaction vessel (there is no headspace purging or cleaning during deprotection).

The resultant peptide is analyzed also as described in Example 1.

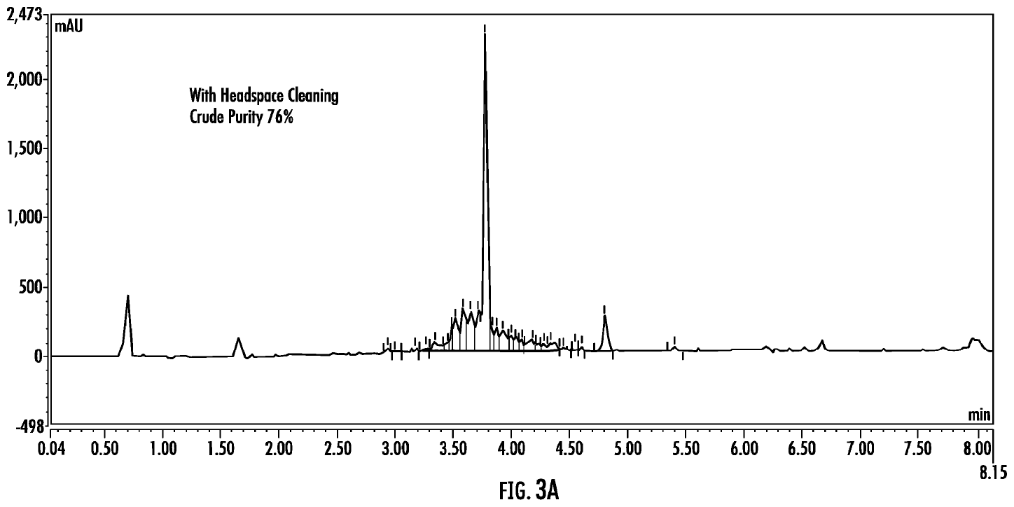

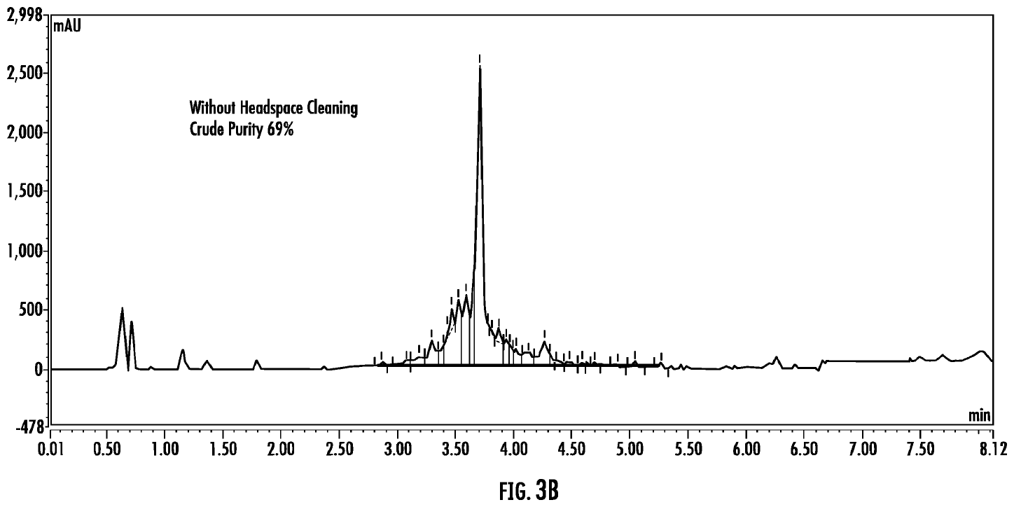

FIGS. 3A and 3B are UPLC chromatographs of thymosin of Example 1 and Comparative Example 1, respectively, and demonstrate an increase in peptide purity using headspace purging (headspace cleaning) during the deprotecting step in accordance with the present disclosure. For example, as indicated in FIG. 3A, thymosin produced using head space purging (headspace cleaning) during the deprotecting step in accordance with the present disclosure exhibits improved purity of 76%. In contrast, as indicated in FIG. 3B, thymosin produced without headspace purging (headspace cleaning) during deprotection exhibits reduced purity of 69%.

### Example 2

Synthesis of Proinsulin with Headspace Cleaning During Deprotection

The peptide Proinsulin FVNQHLKGSH LVEALYLVKG ERGFFYTPKT RREAEDLQVG QVELGGGPGA GSLQPLALEG SLQKRGIVEQ KKTSIKSLYQ LENYKN (MW=9544; 1909 (+5 ion), 1591 (+6 ion), 1364 (+7 ion), 1194 (+8 ion), 1061 (+9 ion), 955 (+10 ion), 868 (+11 ion), 796 (+12 ion), 735 (+13 ion), 682 (+14 ion)) is synthesized under the same conditions described in Example 1, including directing a pressurized nitrogen gas stream continuously through the headspace of the reaction vessel during deprotection (there is headspace purging or cleaning during deprotection).

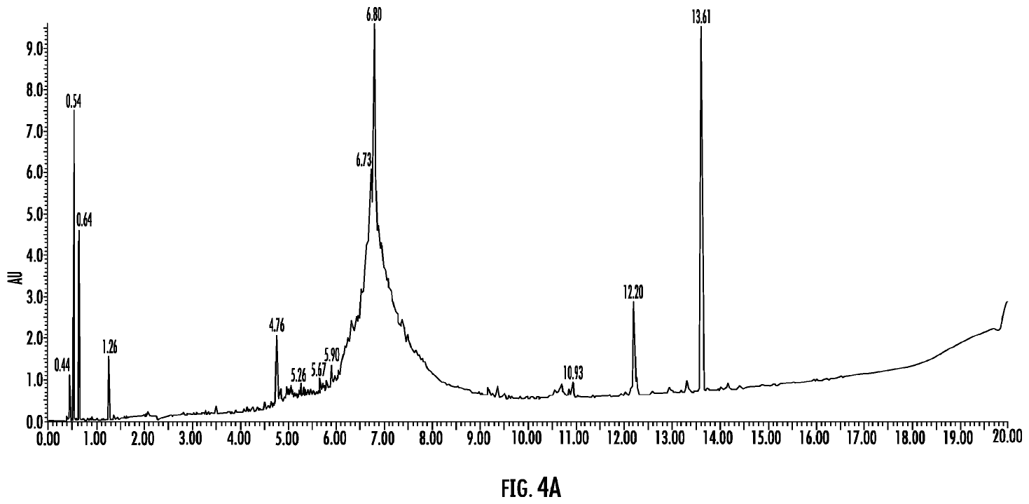

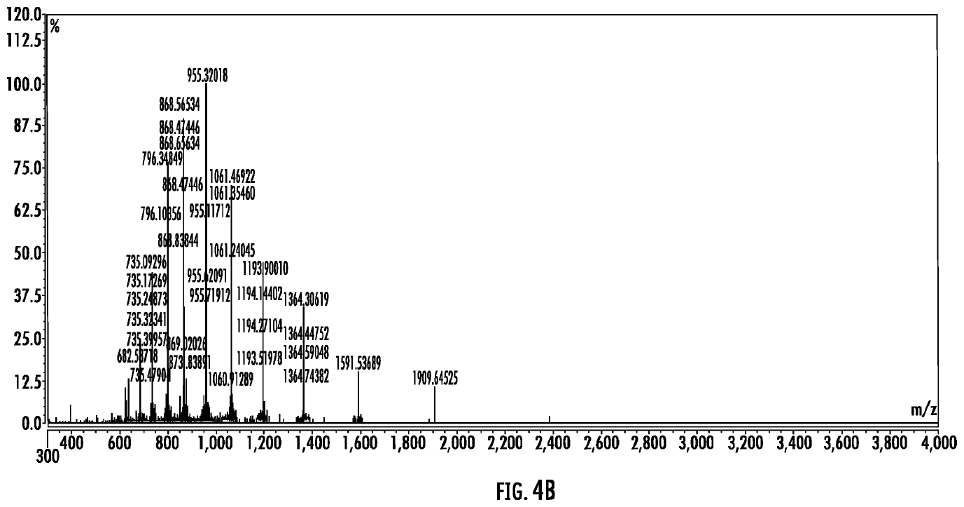

The resultant peptide is analyzed also as described in Example 1. FIGS. 4A and 4B are UPLC chromatograph and mass spectrum, respectively, of proinsulin of Example 2.

### Example 3

Synthesis of HIV-1 Protease with Headspace Cleaning During Deprotection

The peptide HIV-1 Protease PQVTLWQRPI VTIKIGGQLK EALLDTGADD TVLEEMSLPG KWKPKMIGGI GGFIKVRQYD QVSIEIKGHK AIGTVLIGPT PVNIIGRNLL TQLGKTLNF (MW=10775; 1540 (+7 ion), 1347 (+8 ion), 1198 (+9 ion), 1078 (+10 ion), 980 (+11 ion), 898 (+12 ion), 829 (+13 ion), 770 (+14 ion)) is synthesized under the same conditions described in Example 1, including directing a pressurized nitrogen gas stream continuously through the headspace of the reaction vessel during deprotection (there is headspace purging or cleaning during deprotection).

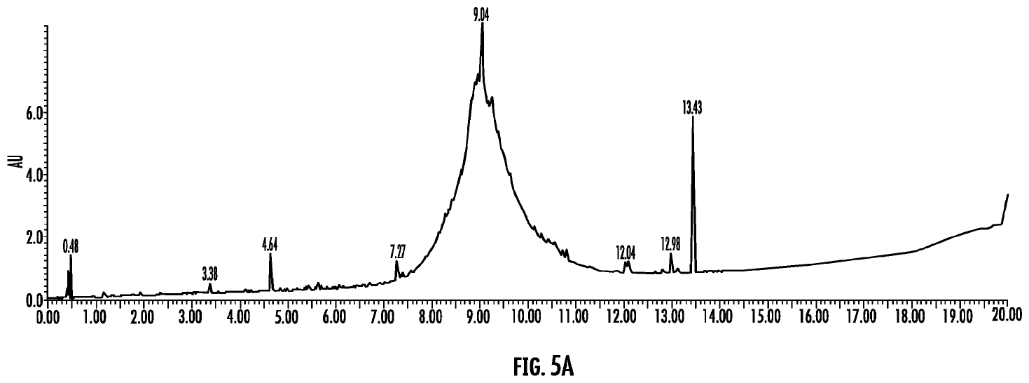

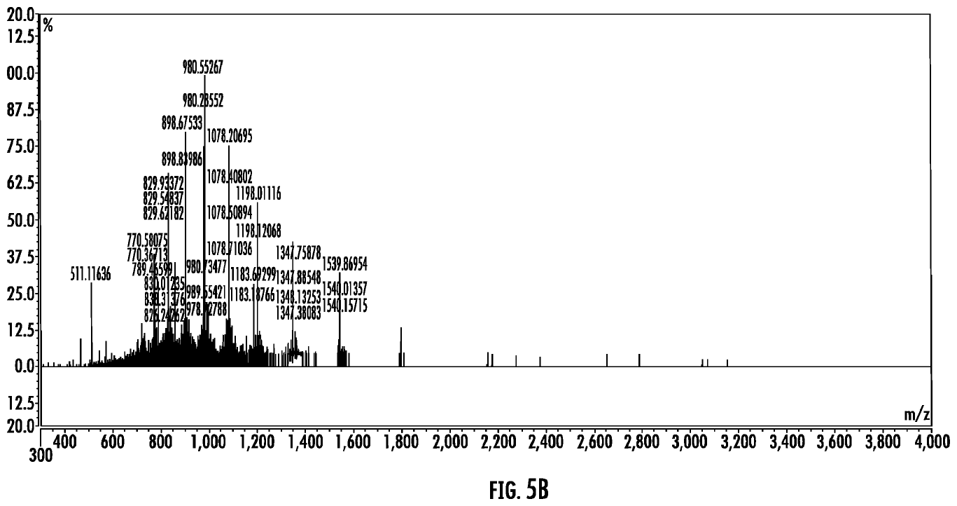

The resultant peptide is analyzed also as described in Example 1. FIGS. 5A and 5B are UPLC chromatograph and mass spectrum, respectively, of HIV-1 Protease of Example 3.

### Example 4

Synthesis of Barstar with Headspace Cleaning During Deprotection

The peptide Barstar KKAVINGEQI RSISDLHQTL KKELALPEYY GENLDALWDK LTGWVEYPLV LEWRQFEQSK QLTENGAESV LQVFREAKAE GKDITIILS (MW=10261; 1711 (+6 ion), 1466 (+7 ion), 1283 (+8 ion), 1141 (+9 ion), 1027 (+10 ion), 933 (+11 ion), 855 (+12 ion)) is synthesized under the same conditions described in Example 1, including directing a pressurized nitrogen gas stream continuously through the headspace of the reaction vessel during deprotection (there is headspace purging or cleaning during deprotection).

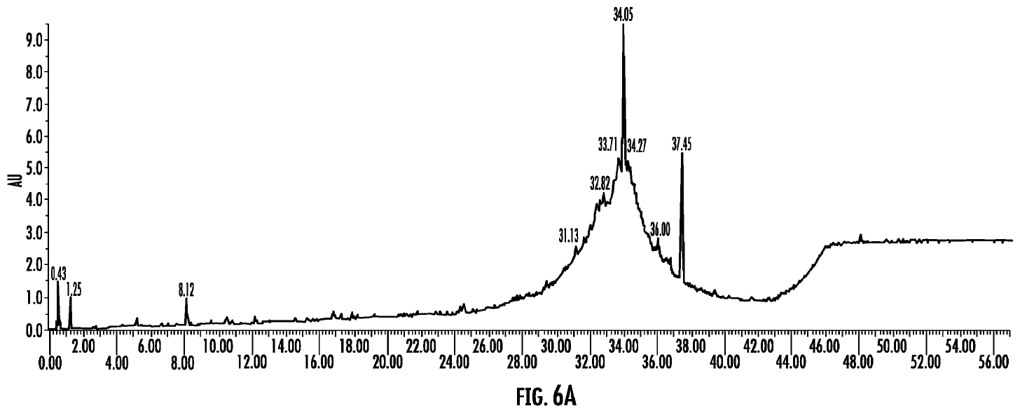

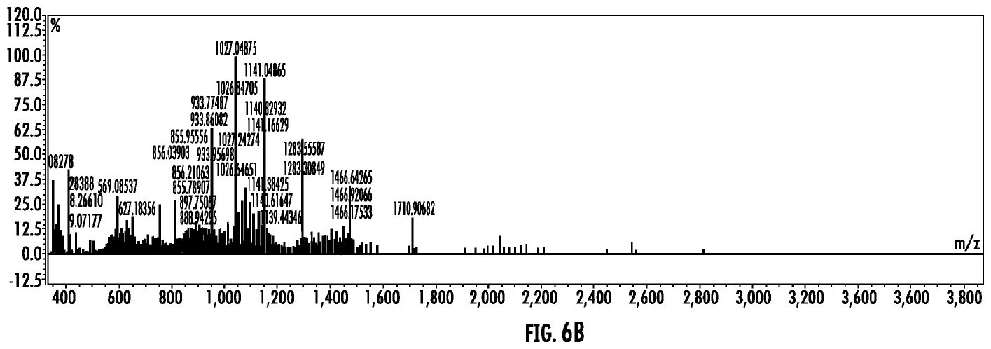

The resultant peptide is analyzed also as described in Example 1. FIGS. 6A and 6B are UPLC chromatograph and mass spectrum, respectively, of Barstar of Example 4.

### Example 5

Synthesis of MDM2 with Headspace Cleaning During Deprotection

The peptide MDM2 MHHHHHHGSM KNTNMSVPTD GAVTTSQIPA SEQETLVRPK PLLLKLLKSV GAQKDTYTMK EVLFYLGQYI MTKRLYDEKQ QHIVYKSNDL LGDLFGVPSF SVKEHRKIYT MIYRNLVVVN QQESSDS (MHHHHHHGSM KNTNMSVPTD GAVTTSQIPA SEQETLVRPK PLLLKLLKSV GAQKDTYTMK EVLFYLGQYI MTKRLYDEKQ QHIVYKSNDL LGDLFGVPSF SVKEHRKIYT MIYRNLVVVN QQESSDS (MW=14607; 1218 (+12 ion), 1125 (+13 ion), 1044 (+14 ion), 975 (+15 ion), 913 (+16 ion), 860 (+17 ion), 813 (+18 ion)) is synthesized under the same conditions described in Example 1, including directing a pressurized nitrogen gas stream continuously through the headspace of the reaction vessel during deprotection (there is headspace purging or cleaning during deprotection).

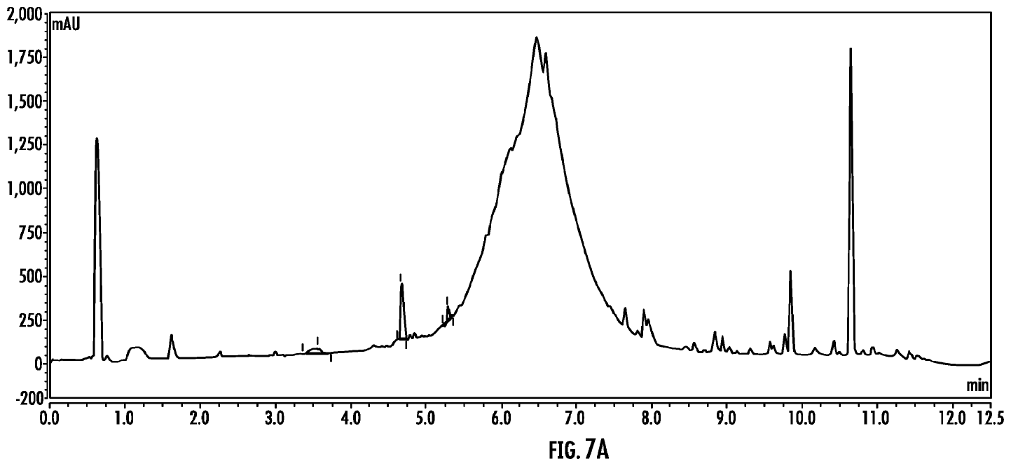

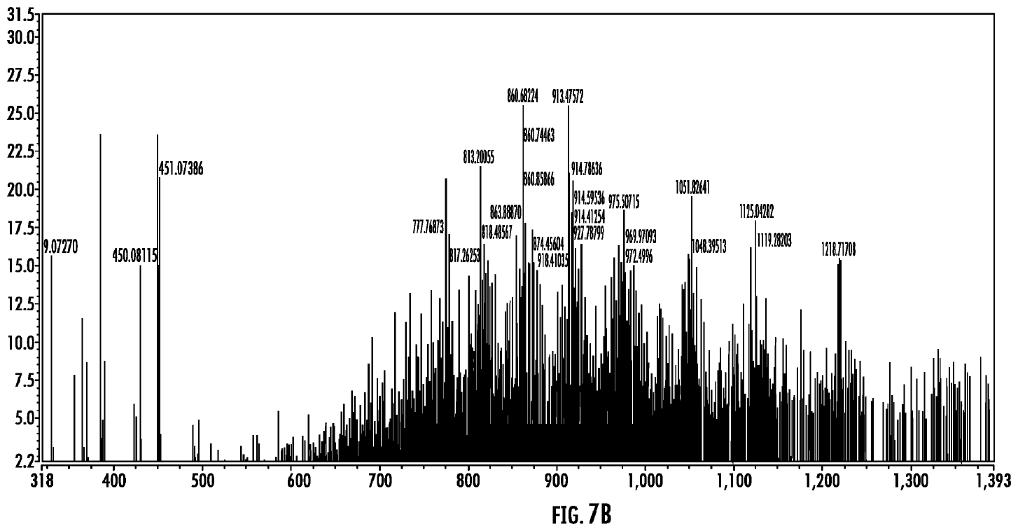

The resultant peptide is analyzed also as described in Example 1. FIGS. 7A and 7B are UPLC chromatograph and mass spectrum, respectively, of MDM2 of Example 5.

### Example 6

Analysis of One-Pot Synthesis of JR 10 mer Using Low Base Concentrations, with and without Post-Deprotection Washing and with and without Headspace Flushing

Examples 1-5 above demonstrate that elevated temperature deprotection steps with microwave SPPS up to 110° C. with inert gas (nitrogen) headspace flushing may result in high purity for even long and difficult sequences. Examples 6 and 7 demonstrate that even using small amounts of deprotecting base may result in essentially complete deprotection and scavenging of the protecting group (e.g., Fmoc protecting group) with only residual base left, which may be small enough to minimize issues for the next coupling step.

JR 10 mer is synthesized using solid phase peptide synthesis using a commercially available automated microwave peptide synthesizer (e.g., from the Liberty line of microwave peptide synthesizers commercially available from CEM Corporation, Matthews, NC, such as Liberty 2.0). PEG-PS resin (e.g., Rink Amide ProTide Resin LL commercially available from CEM Corporation) is used as the solid phase resin support, and coupling reactions are performed in the presence of Fmoc-protected amino acids (AA).

Deprotection reactions are performed by adding a pyrrolidine/dimethylformamide (DMF) deprotection reagent (composition) to an undrained post-coupling mixture. The concentration of pyrrolidine (volume percent pyrrolidine based on the total volume of the deprotection reagent including pyrrolidine and DMF) is noted in Table 1 below. Microwave power is regulated to provide a deprotection temperature and a deprotection reaction time as also noted in Table 1 below.

Table 1 further indicates whether post-deprotection washing and/or headspace flushing is used. For samples in Table 1 wherein “Headspace Flushing” is indicated as “ON,” a nitrogen gas stream is directed through the headspace of the reaction vessel to purge headspace gas from the reaction vessel in accordance with embodiments of the present disclosure described herein (e.g., directing a pressurized nitrogen gas stream into the reaction vessel through an entry port such as shown in FIGS. 1A and 1B, through the headspace, and out of the reaction vessel through an exit port such as a vent port such as shown in FIGS. 1A and 1B). For samples in Table 1 wherein “Headspace Flushing” is indicated as “OFF,” headspace flushing as described herein is not used. For samples in Table 1 wherein “Post-Deprotection Washing” is used, the washing step includes washing two times using 4 mL of DMF after each deprotection step.

Following completion of synthesis of the JR 10 mer, the JR 10 mer is cleaved from the solid phase and crude purity of the resultant JR 10 mer is analyzed. The results are also reported in Table 1 below.

TABLE 1

| | | | | Post- | | |
|---|---|---|---|---|---|---|
| | De- | De- | De- | De- | Head- | |
| | protection | protection | protection | protection | space | Crude |
| Resin | Reagent | Temp. | Time | Washing | Flushing | Purity |
| RA | 4.5% | 110 C. | 40 sec | 2 × 4 mL | ON | 87% |
| ProTide | pyrrolidine/ | | | DMF | | |
| LL | DMF | | | | | |
| RA | 4.5% | 110 C. | 80 sec | None | OFF | 20% |
| ProTide | pyrrolidine/ | | | | | |
| LL | DMF | | | | | |
| RA | 3% | 110 C. | 80 sec | None | OFF | 56% |
| ProTide | pyrrolidine/ | | | | | |
| LL | DMF | | | | | |
| RA | 2% | 110 C. | 80 sec | None | OFF | 49% |
| ProTide | pyrrolidine/ | | | | | |
| LL | DMF | | | | | |
| RA | 3.5% | 110 C. | 80 sec | None | ON | 86% |
| ProTide | pyrrolidine/ | | | | | |
| LL | DMF | | | | | |
| RA | 3% | 110 C. | 80 sec | None | ON | 84% |
| ProTide | pyrrolidine/ | | | | | |
| LL | DMF | | | | | |
| RA | 3% | 110 C. | 80 sec | None | ON | 72% |
| MBHA | pyrrolidine/ | | | | | |
| PS LL | DMF | | | | | |
| RA | 3% | 110 C. | 60 sec | None | ON | 63% |
| ProTide | pyrrolidine/ | | | | | |
| LL | DMF | | | | | |
| RA | 2% | 110 C. | 80 sec | None | ON | 65% |
| ProTide | pyrrolidine/ | | | | | |
| LL | DMF | | | | | |

The results for the JR peptide synthesis show that a high purity result can be obtained without any washing when using the headspace flushing as described herein with each deprotection step. For example, without being bound by any explanation or theory and without limiting the scope of the invention, it is currently believed that directing an inert gas (nitrogen gas) into the reaction vessel through an entry port (such as shown in FIGS. 1A and 1B), through the headspace, and out of the reaction vessel through an exit port (e.g., a vent port such as shown in FIGS. 1A and 1B) may result in both a higher gas exchange rate above the deprotection solution and a top down directional flow which pushes condensation back into the reaction vessel where it is then reheated

In addition, also without being bound by any explanation or theory and without limiting the scope of the invention, it is currently believed that Example 6 demonstrates that pyrrolidine base, which has a lower boiling point (87° C.) compared to piperidine (106° C.), may be significantly evaporated in an Fmoc removal step and that even using small amounts thereof (as low as 2 vol %) may result in essentially complete deprotection and scavenging of the Fmoc group with only residual base left, which may be small enough to minimize issues for the next coupling step. It is also currently believed that this is the first ever demonstrated SPPS process that eliminates all washing steps during the standard cycle and the SPPS process generates only 4.25 mL of total waste for each standard amino acid cycle at the common 0.1 mmol research scale.

### Example 7

Analysis of One-Pot Synthesis of ¹⁻⁴²β-Amyloid and Liraglutide Sequences Using Protecting Composition Having a Low Base Concentration without Post-Deprotection Washing and with Headspace Flushing

Two other well-known difficult sequences, namely, the ¹⁻⁴²β-amyloid and Liraglutide sequences, are next investigated. The sequences are synthesized using solid phase peptide synthesis with a commercially available automated microwave peptide synthesizer (e.g., from the Liberty line of microwave peptide synthesizers commercially available from CEM Corporation, Matthews, NC, such as Liberty 2.0). Fmoc-Gly-Wang-Protide or Fmoc-Ala-Wang-Protide resin is used as the solid phase resin support, and coupling reactions are performed in the presence of Fmoc-protected amino acids (AA).

Deprotection reactions are performed by adding a pyrrolidine/dimethylformamide (DMF) deprotection reagent (composition) to an undrained post-coupling mixture. The concentration of pyrrolidine (volume percent pyrrolidine based on total volume of the deprotection reagent including pyrrolidine and DMF) is noted in Table 2 below. Based on the results of Example 6, a 3 vol % pyrrolidine concentration is chosen as a middle value utilization of process toward the synthesis of these sequences. Microwave power is regulated to provide a deprotection temperature and a deprotection reaction time as also noted in Table 2 below.

Table 2 indicates that no post-deprotection washing is used and that headspace flushing is used. “Headspace Flushing” indicated as “ON” refers to the use of a nitrogen gas stream directed through the headspace of the reaction vessel to purge headspace gas from the reaction vessel in accordance with embodiments of the present disclosure described herein (e.g., directing a pressurized nitrogen gas stream into the reaction vessel through an entry port such as shown in FIGS. 1A and 1B, through the headspace, and out of the reaction vessel through an exit port such as a vent port such as shown in FIGS. 1A and 1B). Following completion of synthesis of the sequences, the sequences are cleaved from the solid phase and crude purity of the resultant sequences is analyzed. The results are reported in Table 2 below.

TABLE 2

| | | De- | De- | De- | | Post- | | |
|---|---|---|---|---|---|---|---|---|
| | | protection | protection | protection | | Deprotection | Headspace | Crude |
| Peptide | Resin | Reagent | Temp. | Time | | Washing | Flushing | Purity |
| Liraglutide | Fmoc- | 3% | 110 C. | 80 | sec | None | ON | 73% |
| | Gly- | pyrrolidine/ | | | | | | |
| | Wang- | DMF | | | | | | |
| | ProTide | | | | | | | |
| Liraglutide | Fmoc- | 3% | 80 C. | 8 | min | None | ON | 75% |
| | Gly- | pyrrolidine/ | | | | | | |
| | Wang- | DMF | | | | | | |
| | ProTide | | | | | | | |
| ¹⁻⁴²β-Amyloid | Fmoc- | 3% | 110 C. | 80 | sec | None | ON | 80% |

| | |
|---|---|
| Ala- | pyrrolidine/ |
| Wang- | DMF |
| ProTide | |

The results for the ⁴²β-amyloid and Liraglutide sequences also show that a high purity result can be obtained without any washing when using the headspace flushing as described herein in each deprotection step.

The foregoing Examples 6 and 7 demonstrate that embodiments of the present disclosure including a deprotection step described herein (e.g., a deprotection step of the second embodiment) can provide an improved process that can eliminate one or more (e.g., all) washing steps for solid phase peptide synthesis (e.g., can eliminate post-deprotection and/or post-coupling washing steps). In some embodiments, the process may use low amounts of deprotecting base (e.g., about 3-4 vol % pyrrolidine) for Fmoc removal; and/or heating (e.g., microwave heating at 80-110° C.); and/or base removal from the deprotection solution with elevated temperatures and/or nitrogen purging (headspace flushing), which may result in a low enough remaining base so as to eliminate the need for washing before the next amino acid is added. Examples 6 and 7 demonstrate significant robustness even on longer, more difficult sequences such as Liraglutide. Thus, the processes may provide a major savings in solvent and time.

In the foregoing, examples of embodiments have been disclosed. The present invention is not limited to such exemplary embodiments. In the foregoing, descriptions of sequences of steps or other actions are described for purposes of providing examples, and not for the purpose of limiting the scope of this disclosure (e.g., where appropriate, steps or actions may be performed in different sequences than described above, and steps and actions may be omitted and/or added). The figures are schematic representations and so are not necessarily drawn to scale. Unless otherwise noted, specific terms have been used in a generic and descriptive sense and not for purposes of limitation.

Numerical values provided throughout this disclosure can be approximate, and for each range specified in this disclosure, all values within the range (including end points) and all subranges within the range are also disclosed. Those of ordinary skill in the art will also readily understand that, in different implementations of the features of this disclosure, reasonably different engineering tolerances, precision, and/or accuracy (for example with respect to numerical value(s)) may be applicable and suitable for obtaining the desired result. Those of ordinary skill will accordingly readily understand the meaning, usage, etc. herein of terms such as “substantially,” “about,” “approximately,” and the like. As non-limiting examples, the term “about” can indicate that a numeric value can vary by plus or minus 25%, for example plus or minus 20%, for example plus or minus 15%, for example plus or minus 10%, for example plus or minus 5%, for example plus or minus 4%, for example plus or minus 3%, for example plus or minus 2%, for example plus or minus 1%, for example plus or minus less than 1%, for example plus or minus 0.5%, for example less than plus or minus 0.5%, including all values and subranges therebetween for each of the above ranges.

As used herein, the phrase “and/or” includes any and all combinations of one or more of the associated listed items (e.g., can refer to elements that are conjunctively present in some embodiments and elements that are disjunctively present in other embodiments), and in some embodiments optionally in combination with other elements not specifically identified by the “and/or” phrase. As non-limiting examples, “A and/or B” can refer in some embodiments to A without B; in some embodiments to B without A; in some embodiments to both A and B; etc.

As used herein, the phrase “at least one” in reference to a list of one or more elements can refer to at least one element selected from any one or more of the elements in the list of elements, but not necessarily including at least one of each and every element specifically listed within the list of elements and not excluding any combinations of elements in the list of elements. In some embodiments, elements may be optionally present other than the elements specifically identified within the list of elements to which the phrase “at least one” refers, whether related or unrelated to those elements specifically identified. As non-limiting examples, “at least one of A and B”; “at least one of A or B”; and/or “at least one of A and/or B” can refer in some embodiments to at least one, optionally including more than one, A, with no B present (and optionally including elements other than B); in some embodiments to at least one, optionally including more than more one, B, with no A present (and optionally including elements other than A); in some embodiments to at least one, optionally including more than one, A, and at least one, optionally including more than one, B (and optionally including other elements); etc.

As used herein, indefinite articles “a” and “an” refer to at least one (“a” and “an” can refer to singular and/or plural element(s)).

1. A process for deprotecting a protected amino acid during solid phase peptide synthesis, comprising:
heating a protected amino acid and a deprotecting agent in a lower interior portion of a reaction vessel during a deprotection reaction removing a protecting group from the protected amino acid, wherein the heating step volatizes deprotecting agent into an upper interior portion of the reaction vessel; and
directing a first inert gas into the upper interior portion of the reaction vessel through a first opening in an upper portion of the reaction vessel and out of the upper interior portion of the reaction vessel through a second opening in the upper portion of the reaction vessel during the heating step to remove volatized deprotecting agent from the interior of the reaction vessel during the deprotection reaction.

2. The process according to claim 1, comprising continuously directing the first inert gas through the upper interior portion of the reaction vessel during the heating step to remove volatized deprotecting agent from the interior of the reaction vessel during the deprotection reaction.

3-8. (canceled)

9. The process according to claim 1, wherein the directing step comprises:
directing the first inert gas through the upper interior portion of the reaction vessel at a first pressure, and
directing a second inert gas into the lower interior portion of the reaction vessel at a second pressure that is lower than the first pressure of the first inert gas.

10-11. (canceled)

12. The process according to claim 9, wherein the first pressure of the first inert gas ranges from about 1 psi to about 25 psi and the second pressure of the second inert gas is less than the first pressure.

13. The process according to claim 1, wherein:
the reaction vessel is a microwave transparent reaction vessel;
the heating step comprises heating the protected amino acid and the deprotecting agent using microwave radiation; and
the heating is conducted at a temperature from about 70° C. to about 120° C.

14. (canceled)

15. The process according to claim 1, wherein the deprotecting agent comprises pyrrolidine.

16-20. (canceled)

21. The process according to claim 1, wherein:
the reaction vessel comprises at least one outer side wall extending around an interior space,
a portion of the volatized deprotecting agent condenses on the side wall, and
the directing step further comprises directing the first inert gas towards the side wall to move condensed deprotecting agent downwardly towards the lower interior portion of the reaction vessel.

22-23. (canceled)

24. A process for solid phase peptide synthesis, comprising:
deprotecting a first protected amino acid to remove a protecting group from the protected amino acid, wherein the deprotecting comprises:
heating the first protected amino acid and a deprotecting agent in a lower interior portion of a reaction vessel during the deprotecting, wherein the heating evaporates deprotecting agent into an upper interior portion of the reaction vessel; and
directing an inert gas into the upper interior portion of the reaction vessel through a first opening in an upper portion of the reaction vessel and out of the upper interior portion of the reaction vessel through a second opening in the upper portion of the reaction vessel during the heating to remove evaporated deprotecting agent from the interior of the reaction vessel during the deprotecting;
coupling a second amino acid to the deprotected amino acid to form a peptide from the first and second amino acids; and
repeating the deprotecting and coupling to form a peptide comprising the first, second, and successive amino acids.

25-34. (canceled)

35. The process according to claim 24, wherein:
the reaction vessel is a microwave transparent reaction vessel;
the heating step comprises heating the protected amino acid and the deprotecting agent using microwave radiation; and
the heating is conducted at a temperature from about 70° C. to about 120° C.

36. (canceled)

37. A process for deprotecting a protected amino acid during solid phase peptide synthesis, comprising:
removing a protecting group of a protected amino acid with a deprotecting composition, the deprotecting composition including a deprotecting agent in an amount of about 5 vol % or less based on the total volume of the deprotecting composition, wherein:
the protected amino acid and the deprotecting composition are present in a lower interior portion of a reaction vessel and are heated during removing of the protecting group, and
at least a portion of the deprotecting agent evaporates into an upper interior portion of the reaction vessel during the removing step; and
directing an inert gas through the interior of the reaction vessel to remove evaporated deprotecting agent from the upper interior portion of the reaction vessel during the step of removing the protecting group.

38. The process according to claim 37, wherein the deprotecting composition includes the deprotecting agent in an amount from about 2 vol % to about 5 vol % based on the total volume of the deprotecting composition.

39. The process according to claim 37, wherein the deprotecting composition includes the deprotecting agent in an amount from about 2 vol % to about 4.5 vol % based on the total volume of the deprotecting composition.

40. The process according to claim 37, wherein the deprotecting composition includes the deprotecting agent in an amount from about 3 vol % to about 4.5 vol % based on the total volume of the deprotecting composition.

41. The process according to claim 37, wherein the directing step comprises introducing the inert gas into the upper interior portion of the reaction vessel through a first opening located in an upper portion of the reaction vessel; and venting the inert gas and evaporated deprotecting agent from the upper interior portion of the reaction vessel through a second opening located in an upper portion of the reaction vessel.

42. The process according to claim 37, wherein the directing step comprises introducing the inert gas into the lower interior portion of the reaction vessel through an opening located in a lower portion of the reaction vessel; and venting the inert gas and evaporated deprotecting agent from the upper interior portion of the reaction vessel through an opening located in an upper portion of the reaction vessel.

43. The process according to claim 37, wherein the directing step comprises introducing the inert gas into both the upper interior portion of the reaction vessel through a first opening located in an upper portion of the reaction vessel and the lower interior portion of the reaction vessel through an opening located in a lower portion of the reaction vessel; and venting the inert gas and evaporated deprotecting agent from the upper interior portion of the reaction vessel.

44-47. (canceled)

48. The process according to claim 37, wherein:
the reaction vessel is a microwave transparent reaction vessel;
the heating step comprises heating the protected amino acid and the deprotecting agent using microwave radiation; and
the heating is conducted at a temperature from about 40° C. to about 120° C.

49-51. (canceled)

52. The process according to claim 37, wherein the deprotecting agent comprises pyrrolidine.

53. (canceled)

54. The process according to claim 37, wherein the directing step comprises continuously directing the inert gas through the interior of the reaction vessel during the step of removing the protecting group.

55-56. (canceled)

57. A process for solid phase peptide synthesis, comprising:
deprotecting a first protected amino acid to provide a deprotected amino acid, the deprotecting comprising:
removing a protecting group of the protected amino acid with a deprotecting composition including a deprotecting agent in an amount of about 5 vol % or less based on the total volume of the deprotecting composition, wherein the protected amino acid and the deprotecting composition are present in a lower interior portion of a reaction vessel, the protected amino acid and the deprotecting composition are heated during the removing of the protecting group, and at least a portion of the deprotecting agent evaporates into an upper interior portion of the reaction vessel during the removing step; and
directing an inert gas through the interior of the reaction vessel to remove evaporated deprotecting agent from the upper interior portion of the reaction vessel during the removing of the protecting group;
coupling a second amino acid to the deprotected amino acid to form a peptide from the first and second amino acids; and
repeating the deprotecting and coupling steps to form a peptide comprising the first, second, and successive plurality of amino acids.

58-59. (canceled)

60. The process according to claim 57, wherein the deprotecting composition includes the deprotecting agent in an amount from about 2 vol % to about 5 vol % based on the total volume of the deprotecting composition.

61. The process according to claim 57, wherein the deprotecting composition includes the deprotecting agent in an amount from about 2 vol % to about 4.5 vol % based on the total volume of the deprotecting composition.

62. The process according to claim 57, wherein the deprotecting composition includes the deprotecting agent in an amount from about 3 vol % to about 4.5 vol % based on the total volume of the deprotecting composition.

63. The process according to claim 57, wherein the directing step comprises introducing the inert gas into the upper interior portion of the reaction vessel through a first opening located in an upper portion of the reaction vessel; and venting the inert gas and evaporated deprotecting agent from the upper interior portion of the reaction vessel through a second opening located in an upper portion of the reaction vessel.

64. The process according to claim 57, wherein the directing step comprises introducing the inert gas into the lower interior portion of the reaction vessel through an opening located in a lower portion of the reaction vessel; and venting the inert gas and evaporated deprotecting agent from the upper interior portion of the reaction vessel through an opening located in an upper portion of the reaction vessel.

65. The process according to claim 57, wherein the directing step comprises introducing the inert gas into both the upper interior portion of the reaction vessel through a first opening located in an upper portion of the reaction vessel and the lower interior portion of the reaction vessel through an opening located in a lower portion of the reaction vessel; and venting the inert gas and evaporated deprotecting agent from the upper interior portion of the reaction vessel.

66-69. (canceled)

70. The process according to claim 57, wherein:
the reaction vessel is a microwave transparent reaction vessel;
the heating step comprises heating the protected amino acid and the deprotecting agent using microwave radiation; and
the heating is conducted at a temperature from about 40° C. to about 120° C.

71-73. (canceled)

74. The process according to claim 37, wherein the deprotecting agent comprises pyrrolidine.

75. (canceled)

76. The process according to claim 57, comprising continuously directing the inert gas through the interior of the reaction vessel during the removing of the protecting group.

77-78. (canceled)

79. The process according to claim 57, wherein the process does not include a washing step after the deprotecting step and before the coupling step.

80. The process according to claim 57, further comprising washing the interior of the reaction vessel after the deprotection step and before the coupling step with solvent in an amount that is less than the total volume of the deprotecting composition.

81. The process according to claim 80, comprising washing the interior of the reaction vessel after the deprotection step and before the coupling step with solvent in an amount that is less than ½ of the total volume of the deprotecting composition.

82. The process according to claim 80, comprising washing the interior of the reaction vessel after the deprotection step and before the coupling step with solvent in an amount that is less than ⅓ of the total volume of the deprotecting composition.

83-103. (canceled)

104. The process according to claim 24, wherein the deprotecting agent comprises pyrrolidine.
